(19) United States
(12) Patent Application Publication
(10) Pub. No.: US 2020/0046758 A1
(43) Pub. Date: Feb. 13, 2020

(54) ORAL CHOLESTYRAMINE FORMULATION AND USE THEREOF

(71) Applicant: Albireo AB, Göteborg (SE)

(72) Inventors: Per-Göran Gillberg, Mölndal (SE); Nils Ove Gustafsson, Löddeköpinge (SE); Nils-Olof Lindberg, Limhamn (SE); Jessica Elversson, Dalby (SE)

(21) Appl. No.: 16/537,393

(22) Filed: Aug. 9, 2019

Related U.S. Application Data
(60) Provisional application No. 62/716,473, filed on Aug. 9, 2018.

(51) Int. Cl. A61K 31/785; A61K 9/16; A61K 9/00; A61K 9/48

(52) U.S. Cl. CPC A61K 31/785; A61K 9/1635; A61K 9/1652; A61K 9/4891; A61K 9/009; A61K 9/4866; A61K 9/167

(57) ABSTRACT

The invention relates to an oral formulation for targeted delivery of cholestyramine to the colon, comprising a plurality of cholestyramine pellets that are coated with a diffusion-controlled inner coating and an enteric outer coating. The invention also relates to the use of this formulation in the treatment of bile acid malabsorption.

### CROSS-REFERENCE TO RELATED APPLICATIONS

This application claims priority to U.S. provisional application No. 62/716,473, filed Aug. 9, 2018, the disclosure of which is incorporated by reference herein in its entirety.

### TECHNICAL FIELD

The invention relates to an oral formulation for targeted delivery of cholestyramine to the colon, comprising a plurality of cholestyramine pellets that are coated with a diffusion-controlled inner coating and an enteric outer coating. The invention also relates to the use of this formulation in the treatment of bile acid malabsorption.

### BACKGROUND

Bile acid malabsorption is a condition characterized by an excess of bile acids in the colon, often leading to chronic diarrhoea. Bile acids are steroid acids that are synthesized and conjugated in the liver. From the liver, they are excreted through the biliary tree into the small intestine where they participate in the solubilisation and absorption of dietary lipids and fat-soluble vitamins. When they reach the ileum, bile acids are reabsorbed into the portal circulation and returned to the liver. A small proportion of the secreted bile acids is not reabsorbed in the ileum and reaches the colon. Here, bacterial action results in deconjugation and dehydroxylation of the bile acids, producing the secondary bile acids deoxycholate and lithocholate.

In the colon, bile acids (in particular the dehydroxylated bile acids chenodeoxycholate and deoxycholate) stimulate the secretion of electrolytes and water. This increases the colonic motility and shortens the colonic transit time. If present in excess, bile acids produce diarrhoea with other gastrointestinal symptoms such as bloating, urgency and faecal incontinence. There have been several recent advances in the understanding of this condition of bile salt or bile acid malabsorption, or BAM (Pattni and Walters, Br. Med. Bull. 2009, vol 92, p. 79-93; Islam and Di Baise, Pract. Gastroenterol. 2012, vol. 36(10), p. 32-44). Dependent on the cause of the failure of the distal ileum to absorb bile acids, bile acid malabsorption may be divided into Type 1, Type 2 and Type 3 BAM.

Diarrhoea may also be the result of high concentrations of bile acid in the large intestine following treatment with drugs that increase the production of bile acids and/or influence the reabsorption of bile acids by the small intestine, such as treatment with ileal bile acid absorption (IBAT) inhibitors.

The current treatment of bile acid malabsorption aims at binding excess bile acids in the gastrointestinal tract, beginning in the proximal part of the small bowel, thereby reducing the secretory actions of the bile acids. For this purpose, cholestyramine is commonly used as the bile acid sequestrant. Cholestyramine (or colestyramine; CAS Number 11041-12-6) is a strongly basic anion-exchange resin that is practically insoluble in water and is not absorbed from the gastrointestinal tract. Instead, it absorbs and combines with the bile acids in the intestine to form an insoluble complex. The complex that is formed upon binding of the bile acids to the resin is excreted in the faeces. The resin thereby prevents the normal reabsorption of bile acids through the enterohepatic circulation, leading to an increased conversion of cholesterol to bile acids to replace those removed from reabsorption. This conversion lowers plasma cholesterol concentrations, mainly by lowering of the low-density lipoprotein (LDL)-cholesterol.

Cholestyramine is also used as hypolipidaemic agents in the treatment of hypercholesterolemia, type II hyperlipoproteinaemia and in type 2 diabetes mellitus. It is furthermore used for the relief of diarrhoea associated with ileal resection, Crohn's disease, vagotomy, diabetic vagal neuropathy and radiation, as well as for the treatment of pruritus in patients with cholestasis.

In the current treatment of hyperlipidaemias and diarrhoea, the oral cholestyramine dose is 12 to 24 g daily, administered as a single dose or in up to 4 divided doses. In the treatment of pruritus, doses of 4 to 8 g are usually sufficient. Cholestyramine may be introduced gradually over 3 to 4 weeks to minimize the gastrointestinal effects. The most common side-effect is constipation, while other gastrointestinal side-effects are bloating, abdominal discomfort and pain, heartburn, flatulence and nausea/vomiting. There is an increased risk for gallstones due to increased cholesterol concentration in bile. High doses may cause steatorrhoea by interference with the gastrointestinal absorption of fats and concomitant decreased absorption of fat-soluble vitamins. Chronic administration may result in an increased bleeding tendency due to hypoprothrombinaemia associated with vitamin K deficiency or may lead to osteoporosis due to impaired calcium and vitamin D absorption. There are also occasional reports of skin rashes and pruritus of the tongue, skin and perianal region. Due to poor taste and texture and the various side effects, >50% of patients discontinue therapy within 12 months.

Another drawback with the current treatment using cholestyramine is that this agent reduces the absorption of other drugs administered concomitantly, such as oestrogens, thiazide diuretics, digoxin and related alkaloids, loperamide, phenylbutazone, barbiturates, thyroid hormones, warfarin and some antibiotics. It is therefore recommended that other drugs should be taken at least 1 hour before or 4 to 6 hours after the administration of cholestyramine. Dose adjustments of concomitantly taken drugs may still be necessary to perform.

In view of these side effects, it would be desirable if cholestyramine could be formulated as a colon release formulation, i.e. for release of the cholestyramine in the proximal part of the colon. Such a formulation may require a lower dose of cholestyramine and should have better properties regarding texture and taste, and may therefore be better tolerated by the patients. More importantly, colonic release of cholestyramine should be devoid of producing interactions with other drugs and should not induce risks for malabsorption of fat and fat-soluble vitamins, while still binding bile acids in order to reduce the increased colonic secretion and motility. For reasons of patient compliance, it would furthermore be desirable if the number of pills to be taken could be kept as low as possible. Each pill should therefore contain as much cholestyramine as possible.

EP 1273307 discloses preparations for preventing bile acid diarrhoea, comprising a bile acid adsorbent coated with a polymer so as to allow the release of the bile acid adsorbent around an area from the lower part of the small intestine to the cecum. It is shown that cholestyramine granules coated with HPMCAS-HF or ethyl cellulose displayed extensive swelling and bursting under conditions simulating the gastric environment.

Jacobsen et al. (Br. Med. J. 1985, vol. 290, p. 1315-1318) describe a study wherein patients who had undergone ileal resection were administered 500 mg cholestyramine tablets coated with cellulose acetate phthalate (12 tablets daily). In five of the 14 patients in this study, the tablets did not disintegrate in the desired place.

Despite progress made in this area, there still is a need for further improved cholestyramine formulations. In particular, there is a need for oral formulations for targeted delivery of cholestyramine to the colon.

### BRIEF DESCRIPTION OF THE DRAWINGS

FIGS. 1A, 1B, and 1C shows the sequestration profiles for formulations A, B, and C in an assay simulating the pH of the stomach and the small intestine. FIG. 1A shows the results for formulations A, B and C during 6 hours at pH 5.5. FIG. 1B shows the results during 2 hours at pH 1 followed by 4 hours at pH 6.8. FIG. 1C shows the results for 2 hours at pH 1 followed by 4 hours at pH 7.4.

FIG. 2 shows the amount of remaining cholic acid (relative to a control sample) vs. incubation time (h) for formulations A, B and C in an in vitro SHIME® assay. The results for a comparative experiment using pure cholestyramine powder is also shown.

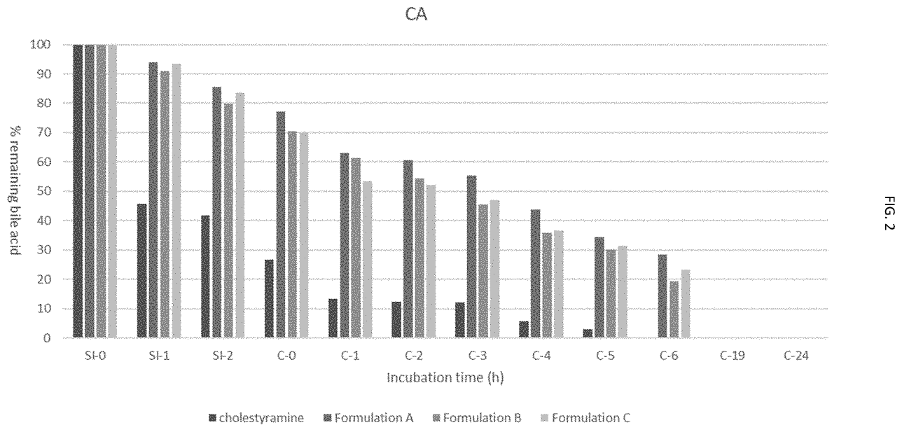

FIG. 3 shows the amount of remaining chenodeoxycholic acid (relative to a control sample) vs. incubation time (h) for formulations A, B and C in an in vitro SHIME® assay. The results for a comparative experiment using pure cholestyramine powder is also shown.

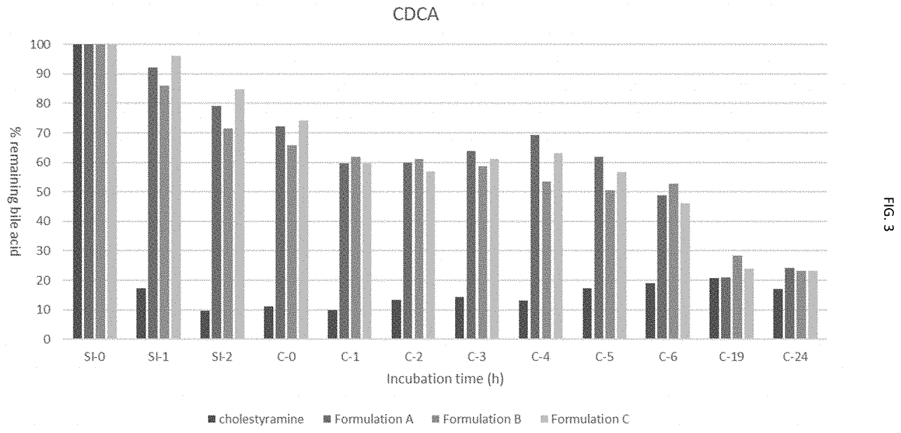

FIG. 4 shows the amount of remaining deoxycholic acid (relative to a control sample) vs. incubation time (h) for formulations A, B and C in an in vitro SHIME® assay. The results for a comparative experiment using pure cholestyramine powder is also shown.

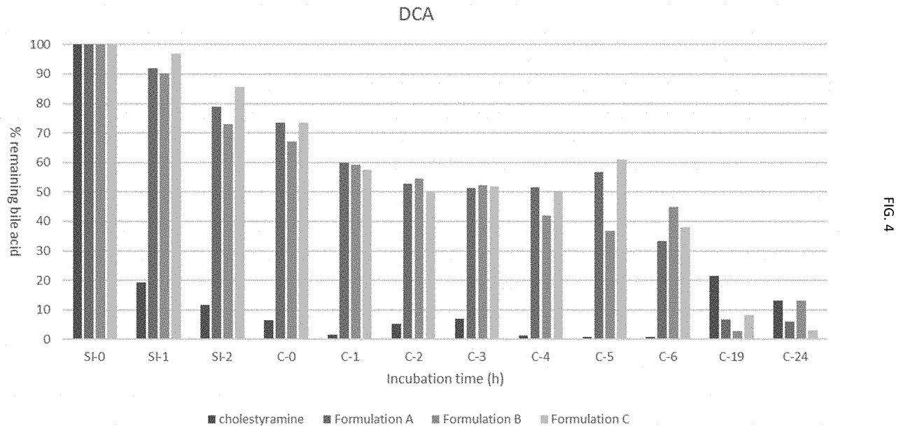

### DETAILED DESCRIPTION OF THE INVENTION

It has been discovered that small and stable pellets of cholestyramine can be obtained, and that these pellets can be coated with a coating layer that prevents release of the pellets until they reach the colon. The combination of small cholestyramine pellets and a colon release coating allows the dose of cholestyramine to be reduced to for example 1.5 g twice daily. It is believed that this dose of cholestyramine is sufficient for binding an excess of bile acids in the colon. The composition disclosed herein further reduces undesired interactions of cholestyramine with other components in the gastrointestinal tract, such as other drugs or nutrients.

In one aspect, the invention relates to an oral formulation for targeted delivery of cholestyramine to the colon, comprising:
a) a plurality of pellets, each pellet comprising cholestyramine and at least about 5% w/w of an acrylate copolymer;
b) a diffusion-controlled inner coating surrounding each pellet; and
c) an enteric outer coating,

and wherein more than about 70% of the cholestyramine is released in the colon.

The coating layers substantially prevent release of cholestyramine from the pellets until they reach the colon.

Preferably, more than about 75% of the cholestyramine is released in the colon, such as more than about 80%, or such as more than about 85%. More preferably, more than about 90% of the cholestyramine is released in the colon.

In another aspect, the invention relates to an oral formulation for targeted delivery of cholestyramine to the colon, comprising:
a) a plurality of pellets, each pellet comprising cholestyramine and at least about 5% w/w of an acrylate copolymer;
b) a diffusion-controlled inner coating surrounding each pellet; and
c) an enteric outer coating,

and wherein less than about 30% of the cholestyramine is released in the small intestine.

Preferably, less than about 25% of the cholestyramine is released in the small intestine, such as less than about 20%, or such as less than about 15%. More preferably, less than about 10% of the cholestyramine is released in the small intestine.

In another aspect, the invention relates to an oral dosage form, comprising:
a) a plurality of pellets, each pellet comprising cholestyramine and at least about 5% w/w of an acrylate copolymer;
b) a diffusion-controlled inner coating surrounding each pellet; and
c) an enteric outer coating;

wherein the oral dosage form exhibits less than about about 30% sequestration of cholic acid, chenodeoxycholic acid, and deoxycholic acid after about 2 hours in small intestinal incubations as measured in the Simulator of the Human Intestinal Microbial Ecosystem (SHIME) model.

In some embodiments, the oral dosage form exhibits less than about 25% sequestration of cholic acid, chenodeoxycholic acid, and deoxycholic acid after about 2 hours in small intestinal incubations as measured in the Simulator of the Human Intestinal Microbial Ecosystem. More preferably, the oral dosage form exhibits less than about 20% sequestration of cholic acid after about 2 hours in small intestinal incubations as measured in the Simulator of the Human Intestinal Microbial Ecosystem (SHIME) model.

The cholestyramine content of the pellets should be as high as possible. The uncoated pellets therefore preferably contain at least about 70% w/w cholestyramine, more preferably at least about 75% w/w cholestyramine, more preferably at least about 80% w/w cholestyramine, even more preferably at least about 85% w/w cholestyramine and most preferably at least about 90% w/w cholestyramine.

In another aspect, the invention relates to an oral formulation for targeted delivery of cholestyramine to the colon, comprising:
a) a plurality of pellets, each pellet comprising cholestyramine and at least about 5% w/w of an acrylate copolymer; and
b) a diffusion-controlled inner coating surrounding each pellet; and
c) an enteric outer coating.

In one embodiment, more than about 70% of the cholestyramine is released in the colon, preferably more than about 75%, such as more than about 80%, or such as more than about 85%. More preferably, more than about 90% of the cholestyramine is released in the colon.

In another embodiment, less than about 30% of the cholestyramine is released in the small intestine, preferably less than about 25%, such as less than about 20%, or such as less than about 15%. More preferably, less than about 10% of the cholestyramine is released in the small intestine.

The presence of specific amounts of a vinylpyrrolidone-based polymer, or of a combination of a vinylpyrrolidone-based polymer and an acrylate copolymer, in the composition of the pellets allows for a high cholestyramine content. The resulting pellets are stable enough to withstand the conditions necessary for applying the coating layers onto the pellets.

The diffusion-controlled inner coating and the enteric outer coating substantially prevent release of cholestyramine from the pellets until they reach the large intestine, in particular the proximal colon. Additionally, the coating prevents the pellets from bursting. When water that diffuses through the coating is absorbed by the cholestyramine, the increasing volume of the cholestyramine leads to swelling of the pellets. The diffusion-controlled inner coating of the pellets is elastic and is therefore able to withstand the swelling of the pellets. The coating thereby prevents burst of the pellets and premature release of the cholestyramine.

Because of its very low solubility, cholestyramine is not “released” from the formulation in that it dissolves from the formulation and diffuses into the intestine. Instead, the cholestyramine probably stays within the gradually degrading structure of the coated pellet. Therefore, as used herein, the term “release” of the cholestyramine refers to the availability of the cholestyramine to the intestinal content in order to bind components (i.e., bile acids) therein.

Pellets

As used herein, the term “pellets” refers to extruded pellets, i.e. pellets obtained through extrusion and spheronization. The preparation of extruded pellets typically comprises the steps of mixing a powder with a liquid to obtain a wet mass, extruding the wet mass, spheronizing the extrudate and drying of the wet pellets.

It is essential that the pellets are stable enough to withstand mechanical stress during handling, such as during drying and coating of the pellets. The stability of the pellets may be expressed in terms of friability, which is the ability of a solid substance (such as a tablet, granule, sphere or pellet) to be reduced to smaller pieces, e.g. by abrasion, breakage or deformation. A low degree of friability means that the solid substance breaks into smaller pieces only to a low extent. As used herein, friability is defined as the reduction in the mass of the pellets occurring when the pellets are subjected to mechanical strain, such as tumbling, vibration, fluidization, etc. Methods for measuring friability are known in the art (e.g., European Pharmacopoeia 8.0, tests 2.9.7 or 2.9.41).

Experiments have shown that the inclusion of smaller amounts of vinylpyrrolidone-based polymer and/or acrylate copolymer than specified above results in lower yield and higher friability of the pellets. Although it is not possible to define acceptable friability limits for pellets in general, friability values of <1.7% w/w friability have been reported as acceptable to withstand stresses associated with fluid bed coating, handling and other processes (Vertommen and Kinget, Drug Dev. Ind. Pharm. 1997, vol. 23, p. 39-46). For the cholestyramine pellets of the present invention, it has been found that a friability of 2.1% is still acceptable. The friability is preferably lower than about 2.5%, more preferably lower than about 2.0%, more preferably lower than about 1.5%, and even more preferably lower than about 1.0%.

The vinylpyrrolidone-based polymer in the pellets may be polyvinylpyrrolidone (povidone) or a vinylpyrrolidone-vinyl acetate copolymer (copovidone). Povidone is a linear, water-soluble polymer made from N-vinylpyrrolidone. Copovidone (also known as copolyvidone) is a linear, water-soluble copolymer of 1-vinyl-2-pyrrolidone (povidone) and vinyl acetate in a ratio of 6:4 by mass. In a preferred embodiment, the vinylpyrrolidone-based polymer is copovidone.

The acrylate copolymer in the pellets may be any pharmaceutically acceptable copolymer comprising acrylate monomers. Examples of acrylate monomers include, but are not limited to, acrylate (acrylic acid), methyl acrylate, ethyl acrylate, methacrylic acid (methacrylate), methyl methacrylate, butyl methacrylate, trimethylammonioethyl methacrylate and dimethylaminoethyl methacrylate. Several acrylate copolymers are known under the trade name Eudragit®.

Poly(ethyl acrylate-co-methyl methacrylate-co-trimethylammonioethyl methacrylate chloride) is a copolymer of ethyl acrylate, methyl methacrylate and a low content of trimethylammonioethyl methacrylate chloride (a methacrylic acid ester with quaternary ammonium groups). The copolymer is also referred to as ammonio methacrylate copolymer. It is insoluble but the presence of the ammonium salts groups makes the copolymer permeable. The copolymer is available as a 1:2:0.2 mixture (Type A) or as a 1:2:0.1 mixture (Type B). 30% aqueous dispersions of Type A and Type B are sold under the trade names Eudragit® RL 30 D and Eudragit® RS 30 D, respectively.

Poly(methyl acrylate-co-methyl methacrylate-co-methacrylic acid) 7:3:1 is a copolymer of methyl acrylate, methyl methacrylate and methacrylic acid. It is insoluble in acidic media but dissolves by salt formation above pH 7.0. A 30% aqueous dispersion is sold under the trade name Eudragit® FS 30 D.

Poly(methacrylic acid-co-ethyl acrylate) 1:1 is a copolymer of ethyl acrylate and methacrylic acid. It is insoluble in acidic media below a pH of 5.5 but dissolves above this pH by salt formation. A 30% aqueous dispersion is sold under the trade name Eudragit® L 30 D-55.

Further suitable acrylate copolymers include poly(ethyl acrylate-co-methyl methacrylate) 2:1, which is a water-insoluble copolymer of ethyl acrylate and methyl methacrylate. 30% aqueous dispersions are sold under the trade names Eudragit® NE 30 D and Eudragit® NM 30 D.

Preferred acrylate copolymers are ammonio methacrylate copolymer, poly(methyl acrylate-co-methyl methacrylate-co-methacrylic acid) 7:3:1, and poly(methacrylic acid-co-ethyl acrylate) 1:1. More preferably, the acrylate polymer is ammonio methacrylate copolymer, and most preferably the acrylate polymer is poly(ethyl acrylate-co-methyl methacrylate-co-trimethylammonioethyl methacrylate chloride) 1:2:0.2.

In one embodiment, the pellets comprise cholestyramine and at least about 5% w/w of an acrylate copolymer.

In a more preferred embodiment, the pellets comprise cholestyramine and at least about 5% w/w of an ammonio methacrylate copolymer.

In some embodiments, the pellets comprise at least about 70% w/w cholestyramine. In some embodiments, the pellets comprise at least about 75% w/w cholestyramine. In some embodiments, the pellets comprise at least about 80% w/w cholestyramine. In some embodiments, the pellets comprise at least about 85% w/w cholestyramine.

The pellets may further comprise an excipient such as microcrystalline cellulose. In one embodiment, the pellets comprise from about 0 to about 20% w/w microcrystalline cellulose, such as from about 0 to about 10% w/w microcrystalline cellulose, or such as from about 5 to 15% w/w microcrystalline cellulose. In a more preferred embodiment, the pellets comprise from about 0 to about 5% w/w microcrystalline cellulose.

In another embodiment, the pellets are free from microcrystalline cellulose.

In some embodiments, the pellets comprise cholestyramine and at least about 5% w/w of an acrylate copolymer, such as at least about 6% w/w of an acrylate copolymer, or such as at least about 7% w/w of an acrylate copolymer, or such as at least about 8% w/w of an acrylate copolymer. The acrylate copolymer is preferably ammonio methacrylate copolymer. Without being bound by any theory, it is believed that a higher acrylate copolymer content may improve the extrusion and spheronization process, and lead to more spherical shaped pellets.

In some embodiments, the pellets further comprise a vinylpyrrolidone-based polymer, such as at least about 5% w/w, such as at least about 6% w/w, such as at least about 7% w/w, such as at least about 8% w/w, such as at least about 9% w/w, or such as at least about 10% w/w of a vinylpyrrolidone-based polymer.

In some embodiments, the pellets comprise cholestyramine, and a combination of at least about 5% w/w of an acrylate copolymer, and at least about 5% w/w of a vinylpyrrolidone-based polymer. In some embodiments, the pellets comprise cholestyramine, and a combination of at least about 5% w/w of an acrylate copolymer, and at least about 6% w/w of a vinylpyrrolidone-based polymer.

In some embodiments, the pellets comprise cholestyramine, at least about 5% w/w of an acrylate copolymer, and at least about 5% w/w of a vinylpyrrolidone-based polymer. In some embodiments, the pellets comprise cholestyramine, at least about 5% w/w of an acrylate copolymer, and at least about 6% w/w of a vinylpyrrolidone-based polymer.

In another embodiment, the pellets comprise about 80% w/w cholestyramine, about 7.5% w/w of a vinylpyrrolidone-based polymer, about 8% w/w of an acrylate copolymer, and about 4.5% w/w microcrystalline cellulose. More preferably, the pellets comprise about 80% w/w cholestyramine, about 7.5% w/w copovidone, about 8% w/w ammonio methacrylate copolymer, and about 4.5% w/w microcrystalline cellulose.

In some embodiments, the pellets comprise from 70 to 92% w/w cholestyramine, from 6 to 12% w/w of a vinylpyrrolidone-based polymer, at least about 5% w/w of an acrylate copolymer, and from 0 to 20% w/w microcrystalline cellulose. More preferably, the pellets comprise from 80 to 92% w/w cholestyramine, from 6 to 12% w/w of a vinylpyrrolidone-based polymer, at least about 5% w/w of an acrylate copolymer, and from 0 to 5% w/w microcrystalline cellulose.

In some embodiments, the pellets comprise from 70 to 92% w/w cholestyramine, from 6 to 12% w/w of a vinylpyrrolidone-based polymer, about 5% to about 10% w/w of an acrylate copolymer, and from 0 to 20% w/w microcrystalline cellulose. More preferably, the pellets comprise from 80 to 92% w/w cholestyramine, from 6 to 12% w/w of a vinylpyrrolidone-based polymer, about 6% to about 9% w/w of an acrylate copolymer, and from 0 to 5% w/w microcrystalline cellulose.

In some embodiments, the pellets comprise from 70 to 92% w/w cholestyramine, from 6 to 12% w/w copovidone, at least about 5% w/w ammonio methacrylate copolymer, and from 0 to 20% w/w microcrystalline cellulose. More preferably, the pellets comprise from 80 to 92% w/w cholestyramine, from 6 to 12% w/w copovidone, at least about 5% w/w ammonio methacrylate copolymer, and from 0 to 5% w/w microcrystalline cellulose.

In some embodiments, the pellets comprise from 70 to 92% w/w cholestyramine, from 6 to 12% w/w copovidone, about 5% to about 10% w/w ammonio methacrylate copolymer, and from 0 to 20% w/w microcrystalline cellulose. More preferably, the pellets comprise from 80 to 92% w/w cholestyramine, from 6 to 12% w/w copovidone, about 6% to about 9% w/w ammonio methacrylate copolymer, and from 0 to 5% w/w microcrystalline cellulose.

The uncoated pellets rapidly disintegrate under aqueous conditions. However, they are stable enough to withstand the conditions necessary for applying the colon release coating onto the pellets.

In another aspect, the invention relates to an oral formulation, comprising:
a) a plurality of pellets, each pellet comprising cholestyramine and at least about 5% w/w of an acrylate copolymer; and
b) a diffusion-controlled inner coating surrounding each pellet; and
c) an enteric outer coating,

wherein the formulation is capable of releasing more than about 70% of the cholestyramine in the colon.

In some embodiments, each pellet comprises cholestyramine and a combination of at least about 5% w/w of an acrylate copolymer and at least about 5% w/w of a vinylpyrrolidone-based polymer. In some embodiments, each pellet comprises cholestyramine and a combination of at least about 5% w/w of an acrylate copolymer and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, each pellet comprises cholestyramine, at least about 5% w/w of an acrylate copolymer, and at least about 5% w/w of a vinylpyrrolidone-based polymer. In some embodiments, each pellet comprises cholestyramine, at least about 5% w/w of an acrylate copolymer, and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer, and about 4.5% w/w microcrystalline cellulose.

In some embodiments, more than about 75% of the cholestyramine is released in the colon. In other embodiments, more than about 80% of the cholestyramine is released in the colon. In other embodiments, more than about 85% of the cholestyramine is released in the colon. In yet other embodiments, more than about 90% of the cholestyramine is released in the colon.

In some embodiments, the invention relates to an oral formulation, comprising:
a) a plurality of pellets, each pellet comprising cholestyramine and at least about 5% w/w of an acrylate copolymer; and
b) a diffusion-controlled inner coating surrounding each pellet; and
c) an enteric outer coating,

wherein more than 70% of the cholestyramine is released in the colon.

In some embodiments, each pellet comprises cholestyramine and a combination of at least about 5% w/w of an acrylate copolymer and at least about 5% w/w of a vinylpyrrolidone-based polymer. In some embodiments, each pellet comprises cholestyramine and a combination of at least about 5% w/w of an acrylate copolymer and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, each pellet comprises cholestyramine, at least about 5% w/w of an acrylate copolymer, and at least about 5% w/w of a vinylpyrrolidone-based polymer. In some embodiments, each pellet comprises cholestyramine, at least about 5% w/w of an acrylate copolymer, and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer, and about 4.5% w/w microcrystalline cellulose.

In some embodiments, more than about 75% of the cholestyramine is released in the colon. In other embodiments, more than about 80% of the cholestyramine is released in the colon. In other embodiments, more than about 85% of the cholestyramine is released in the colon. In yet other embodiments, more than about 90% of the cholestyramine is released in the colon.

In yet another aspect, the invention relates to an oral formulation, comprising:
a) a plurality of pellets, each pellet comprising cholestyramine and at least about 5% w/w of an acrylate copolymer; and
b) a diffusion-controlled inner coating around each pellet; and
c) an enteric outer coating,

wherein the formulation is capable of releasing less than about 30% of the cholestyramine in the small intestine.

In some embodiments, each pellet comprises cholestyramine and a combination of at least about 5% w/w of an acrylate copolymer and at least about 5% w/w of a vinylpyrrolidone-based polymer. In some embodiments, each pellet comprises cholestyramine and a combination of at least about 5% w/w of an acrylate copolymer and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, each pellet comprises cholestyramine, at least about 5% w/w of an acrylate copolymer, and at least about 5% w/w of a vinylpyrrolidone-based polymer. In some embodiments, each pellet comprises cholestyramine, at least about 5% w/w of an acrylate copolymer, and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer, and about 4.5% w/w microcrystalline cellulose.

In some embodiments, less than about 25% of the cholestyramine is released in the small intestine. In other embodiments, less than about 20% of the cholestyramine is released in the small intestine. In other embodiments, less than about 15% of the cholestyramine is released in the small intestine. In yet other embodiments, less than about 10% of the cholestyramine is released in the small intestine.

In some embodiments, the invention relates to an oral formulation, comprising:
a) a plurality of pellets, each pellet comprising cholestyramine and at least about 5% w/w of an acrylate copolymer; and
b) a diffusion-controlled inner coating around each pellet; and
c) an enteric outer coating,

wherein less than about 30% of the cholestyramine is released in the small intestine.

In some embodiments, each pellet comprises cholestyramine and a combination of at least about 5% w/w of an acrylate copolymer and at least about 5% w/w of a vinylpyrrolidone-based polymer. In some embodiments, each pellet comprises cholestyramine and a combination of at least about 5% w/w of an acrylate copolymer and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, each pellet comprises cholestyramine, at least about 5% w/w of an acrylate copolymer, and at least about 5% w/w of a vinylpyrrolidone-based polymer. In some embodiments, each pellet comprises cholestyramine, at least about 5% w/w of an acrylate copolymer, and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer, and about 4.5% w/w microcrystalline cellulose.

In some embodiments, less than about 25% of the cholestyramine is released in the small intestine. In other embodiments, less than about 20% of the cholestyramine is released in the small intestine. In other embodiments, less than about 15% of the cholestyramine is released in the small intestine. In yet other embodiments, less than about 10% of the cholestyramine is released in the small intestine.

In another aspect, the invention relates to an oral formulation, comprising:
a) a plurality of pellets, each pellet comprising cholestyramine and at least about 5% w/w of an acrylate copolymer; and
b) a diffusion-controlled inner coating surrounding each pellet; and
c) an enteric outer coating, wherein the coating is capable of targeting release of the cholestyramine in the colon,

wherein the pellets exhibit a friability of less than about 2.5% as measured using the European Pharmacopoeia 8.0, test 2.9.7.

In some embodiments, each pellet comprises cholestyramine and a combination of at least about 5% w/w of an acrylate copolymer and at least about 5% w/w of a vinylpyrrolidone-based polymer. In some embodiments, each pellet comprises cholestyramine and a combination of at least about 5% w/w of an acrylate copolymer and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, each pellet comprises cholestyramine, at least about 5% w/w of an acrylate copolymer, and at least about 5% w/w of a vinylpyrrolidone-based polymer. In some embodiments, each pellet comprises cholestyramine, at least about 5% w/w of an acrylate copolymer, and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, the pellets exhibit a friability of less than about 2.0%. In other embodiments, the pellets exhibit a friability of less than about 1.5%. In other embodiments, the pellets exhibit a friability of less than about 1.0%. In yet other embodiments, the pellets exhibit a friability of less than about 0.5%.

In another aspect, the invention relates to an oral formulation, comprising:
a) a plurality of pellets, each pellet comprising cholestyramine and at least about 5% w/w of an acrylate copolymer; and
b) a diffusion-controlled inner coating surrounding each pellet; and
c) an enteric outer coating, wherein the coating is capable of targeting release of the cholestyramine in the colon,

wherein less than about 30% of the cholestyramine is released after about 6 hours at pH of about 5.5 as measured using the USP Dissolution Apparatus 2 (paddle) Ph. Eur. 2.9.3.

In some embodiments, each pellet comprises cholestyramine and a combination of at least about 5% w/w of an acrylate copolymer and at least about 5% w/w of a vinylpyrrolidone-based polymer. In some embodiments, each pellet comprises cholestyramine and a combination of at least about 5% w/w of an acrylate copolymer and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, each pellet comprises cholestyramine, at least about 5% w/w of an acrylate copolymer, and at least about 5% w/w of a vinylpyrrolidone-based polymer. In some embodiments, each pellet comprises cholestyramine, at least about 5% w/w of an acrylate copolymer, and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, less than about 25% of the cholestyramine is released after about 6 hours at pH of about 5.5 as measured using the USP Dissolution Apparatus 2 (paddle) Ph. Eur. 2.9.3. In other embodiments, less than about 20% of the cholestyramine is released after about 6 hours at pH of about 5.5 as measured using the USP Dissolution Apparatus 2 (paddle) Ph. Eur. 2.9.3. In other embodiments, less than about 15% of the cholestyramine is released after about 6 hours at pH of about 5.5 as measured using the USP Dissolution Apparatus 2 (paddle) Ph. Eur. 2.9.3. In yet other embodiments, less than about 10% of the cholestyramine is released after about 6 hours at pH of about 5.5 as measured using the USP Dissolution Apparatus 2 (paddle) Ph. Eur. 2.9.3.

In another aspect, the invention relates to an oral formulation, comprising:
a) a plurality of pellets, each pellet comprising cholestyramine and at least about 5% w/w of an acrylate copolymer; and
b) a diffusion-controlled inner coating surrounding each pellet; and
c) an enteric outer coating, wherein the coating is capable of targeting release of the cholestyramine in the colon,

wherein the formulation exhibits less than about 30% sequestration of cholic acid after about 6 hours at pH of about 5.5 as measured using a USP Dissolution Apparatus 2 (paddle) Ph. Eur. 2.9.3.

In some embodiments, each pellet comprises cholestyramine and a combination of at least about 5% w/w of an acrylate copolymer and at least about 5% w/w of a vinylpyrrolidone-based polymer. In some embodiments, each pellet comprises cholestyramine and a combination of at least about 5% w/w of an acrylate copolymer and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, each pellet comprises cholestyramine, at least about 5% w/w of an acrylate copolymer, and at least about 5% w/w of a vinylpyrrolidone-based polymer. In some embodiments, each pellet comprises cholestyramine, at least about 5% w/w of an acrylate copolymer, and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, the formulation exhibits less than about 25% sequestration of cholic acid after about 6 hours at pH of about 5.5 as measured using a USP Dissolution Apparatus 2 (paddle) Ph. Eur. 2.9.3. In other embodiments, the formulation exhibits less than about 20% sequestration of cholic acid after about 6 hours at pH of about 5.5 as measured using a USP Dissolution Apparatus 2 (paddle) Ph. Eur. 2.9.3. In yet other embodiments, the formulation exhibits less than about 15% sequestration of cholic acid after about 6 hours at pH of about 5.5 as measured using a USP Dissolution Apparatus 2 (paddle) Ph. Eur. 2.9.3.

In another aspect, the invention relates to an oral formulation, comprising:
a) a plurality of pellets, each pellet comprising cholestyramine and at least about 5% w/w of an acrylate copolymer; and
b) a diffusion-controlled inner coating surrounding each pellet; and
c) an enteric outer coating, wherein the coating is capable of targeting release of the cholestyramine in the colon,

wherein the formulation exhibits greater than about 30% sequestration of cholic acid after about 2 hours at pH of about 1 followed by about 4 hours at pH of about 6.8 as measured using a USP Dissolution Apparatus 2 (paddle) Ph. Eur. 2.9.3.

In some embodiments, each pellet comprises cholestyramine and a combination of at least about 5% w/w of an acrylate copolymer and at least about 5% w/w of a vinylpyrrolidone-based polymer. In some embodiments, each pellet comprises cholestyramine and a combination of at least about 5% w/w of an acrylate copolymer and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, each pellet comprises cholestyramine, at least about 5% w/w of an acrylate copolymer, and at least about 5% w/w of a vinylpyrrolidone-based polymer. In some embodiments, each pellet comprises cholestyramine, at least about 5% w/w of an acrylate copolymer, and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, the formulation exhibits greater than about 35% sequestration of cholic acid after about 2 hours at pH of about 1 followed by about 4 hours at pH of about 6.8 as measured using a USP Dissolution Apparatus 2 (paddle) Ph. Eur. 2.9.3. In other embodiments, the formulation exhibits greater than about 40% sequestration of cholic acid after about 2 hours at pH of about 1 followed by about 4 hours at pH of about 6.8 as measured using a USP Dissolution Apparatus 2 (paddle) Ph. Eur. 2.9.3. In yet other embodiments, the formulation exhibits greater than about 45% sequestration of cholic acid after about 2 hours at pH of about 1 followed by about 4 hours at pH of about 6.8 as measured using a USP Dissolution Apparatus 2 (paddle) Ph. Eur. 2.9.3. In yet other embodiments, the formulation exhibits greater than about 50% sequestration of cholic acid after about 2 hours at pH of about 1 followed by about 4 hours at pH of about 6.8 as measured using a USP Dissolution Apparatus 2 (paddle) Ph. Eur. 2.9.3.

In another aspect, the invention relates to an oral formulation, comprising:
a) a plurality of pellets, each pellet comprising cholestyramine and at least about 5% w/w of an acrylate copolymer; and
b) a diffusion-controlled inner coating surrounding each pellet; and
c) an enteric outer coating, wherein the coating is capable of targeting release of the cholestyramine in the colon,

wherein the formulation exhibits less than 30% sequestration of cholic acid after about 2 hours at pH of about 1 as measured using a USP Dissolution Apparatus 2 (paddle) Ph. Eur. 2.9.3.

In some embodiments, each pellet comprises cholestyramine and a combination of at least about 5% w/w of an acrylate copolymer and at least about 5% w/w of a vinylpyrrolidone-based polymer. In some embodiments, each pellet comprises cholestyramine and a combination of at least about 5% w/w of an acrylate copolymer and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, each pellet comprises cholestyramine, at least about 5% w/w of an acrylate copolymer, and at least about 5% w/w of a vinylpyrrolidone-based polymer. In some embodiments, each pellet comprises cholestyramine, at least about 5% w/w of an acrylate copolymer, and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, the formulation exhibits less than about 25% sequestration of cholic acid after about 2 hours at pH of about 1 as measured using a USP Dissolution Apparatus 2 (paddle) Ph. Eur. 2.9.3. In other embodiments, the formulation exhibits less than about 20% sequestration of cholic acid after about 2 hours at pH of about 1 as measured using a USP Dissolution Apparatus 2 (paddle) Ph. Eur. 2.9.3. In other embodiments, the formulation exhibits less than about 15% sequestration of cholic acid after about 2 hours at pH of about 1 as measured using a USP Dissolution Apparatus 2 (paddle) Ph. Eur. 2.9.3. In yet other embodiments, the formulation exhibits less than about 10% sequestration of cholic acid after about 2 hours at pH of about 1 as measured using a USP Dissolution Apparatus 2 (paddle) Ph. Eur. 2.9.3.

In yet another aspect, the invention relates to an oral formulation, comprising:
a) a plurality of pellets, each pellet comprising cholestyramine and at least about 5% w/w of an acrylate copolymer; and
b) a diffusion-controlled inner coating surrounding each pellet; and
c) an enteric outer coating, wherein the coating is capable of targeting release of the cholestyramine in the colon,

wherein the formulation exhibits greater than about 30% sequestration of cholic acid after about 2 hours at pH of about 1 followed by about 4 hours at pH of about 7.4 as measured using a USP Dissolution Apparatus 2 (paddle) Ph. Eur. 2.9.3.

In some embodiments, each pellet comprises cholestyramine and a combination of at least about 5% w/w of an acrylate copolymer and at least about 5% w/w of a vinylpyrrolidone-based polymer. In some embodiments, each pellet comprises cholestyramine and a combination of at least about 5% w/w of an acrylate copolymer and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, each pellet comprises cholestyramine, at least about 5% w/w of an acrylate copolymer, and at least about 5% w/w of a vinylpyrrolidone-based polymer. In some embodiments, each pellet comprises cholestyramine, at least about 5% w/w of an acrylate copolymer, and at least about 6% w/w of a vinylpyrrolidone-based polymer. In some embodiments, each pellet comprises about 80% w/w cholestyramine, about 7.5% w/w of a vinylpyrrolidone-based polymer, about 8% w/w of an acrylate copolymer and about 4.5% w/w microcrystalline cellulose.

In some embodiments, the formulation exhibits greater than about 35% sequestration of cholic acid after about 2 hours at pH of about 1 followed by about 4 hours at pH of about 7.4 as measured using a USP Dissolution Apparatus 2 (paddle) Ph. Eur. 2.9.3. In other embodiments, the formulation exhibits greater than about 40% sequestration of cholic acid after about 2 hours at pH of about 1 followed by about 4 hours at pH of about 7.4 as measured using a USP Dissolution Apparatus 2 (paddle) Ph. Eur. 2.9.3. In other embodiments, the formulation exhibits greater than about 45% sequestration of cholic acid after about 2 hours at pH of about 1 followed by about 4 hours at pH of about 7.4 as measured using a USP Dissolution Apparatus 2 (paddle) Ph. Eur. 2.9.3. In yet other embodiments, the formulation exhibits greater than about 50% sequestration of cholic acid after about 2 hours at pH of about 1 followed by about 4 hours at pH of about 7.4 as measured using a USP Dissolution Apparatus 2 (paddle) Ph. Eur. 2.9.3.

Diffusion-Controlled Coating

The diffusion-controlled inner coating provides a modified release of the cholestyramine, i.e. the cholestyramine is not made available at once but over an extended period of time. The coating comprises one or more polymers that are insoluble at any pH value, but that are permeable to water and small molecules dissolved therein. Examples of such polymers include, but are not limited to, poly(ethyl acrylate-co-methyl methacrylate-co-trimethylammonioethyl methacrylate chloride) 1:2:0.2 (Eudragit® RL 30 D), poly(ethyl acrylate-co-methyl methacrylate-co-trimethylammonioethyl methacrylate chloride) 1:2:0.1 (Eudragit® RS 30 D), poly(ethyl acrylate-co-methyl methacrylate) 2:1 (Eudragit® NE 30 D or Eudragit® NM 30 D) and polyvinyl acetate (Kollicoat® SR 30 D). The diffusion-controlled inner coating preferably comprises poly(ethyl acrylate-co-methyl methacrylate-co-trimethylammonioethyl methacrylate chloride) 1:2:0.2 (Eudragit® RL 30 D), poly(ethyl acrylate-co-methyl methacrylate-co-trimethylammonioethyl methacrylate chloride) 1:2:0.1 (Eudragit® RS 30 D) or a combination thereof, and most preferably poly(ethyl acrylate-co-methyl methacrylate-co-trimethylammonioethyl methacrylate chloride) 1:2:0.1.

When water is absorbed by the cholestyramine, the increasing volume of the cholestyramine leads to swelling of the pellets. The diffusion-controlled inner coating should therefore be elastic (i.e., have high elongation at break). Because of the elasticity of the coating, the coating is able to withstand this swelling. Burst of the pellets and premature release of the cholestyramine is thereby avoided.

The elasticity of the coating may be the result of the elasticity of the organic polymer(s) itself, or may be induced by the addition of a plasticizer. Examples of suitable plasticizers include triethyl citrate, glyceryl triacetate, tributyl citrate, diethyl phthalate, acetyl tributyl citrate, dibutyl phthalate and dibutyl sebacate.

Enteric Coating

The enteric coating comprises a pH-sensitive polymer that is stable and insoluble at the acidic pH values found in the stomach (pH ˜1-3) but that breaks down rapidly or becomes soluble at less acidic pH values, such as the pH values found in the small intestine (pH ˜6 to 7). Examples of such pH-sensitive polymers include, but are not limited to, cellulose acetate phthalate, cellulose acetate succinate, hydroxypropyl methylcellulose acetate succinate, hydroxypropyl methylcellulose phthalate, poly(methacrylic acid-co-methyl methacrylate) 1:1 (Eudragit® L 100), poly(methacrylic acid-co-methyl methacrylate) 1:2 (Eudragit® S 100), poly(methacrylic acid-co-ethyl acrylate) 1:1 (Eudragit® L 100-55), poly(methyl acrylate-co-methyl methacrylate-co-methacrylic acid) 7:3:1 (Eudragit® FS 30 D), polyvinyl acetate phthalate, shellac, sodium alginate, and zein, as well as mixtures thereof. The enteric coating preferably comprises a pH-sensitive polymer selected from the group consisting of poly(methacrylic acid-co-methyl methacrylate) 1:1, hydroxypropyl methylcellulose acetate succinate and poly(methacrylic acid-co-methyl methacrylate) 1:2. The enteric coating most preferably comprises hydroxypropyl methylcellulose acetate succinate.

The diffusion controlled and enteric coatings may comprise one or more additives, such as acids and bases, plasticizers, glidants, and surfactants. Examples of suitable acids include organic acids such as citric acid, acetic acid, trifluoroacetic add, propionic acid, succinic acid, glycolic add, lactic add, mac add, tartaric acid, ascorbic acid, pamoic add, maleic add, hydroxymaleic add, phenylacetic acid, glutamic add, benzoic acid, salicylic: add, mesylic add, esylic acid, besylic add, sulfanilic add, 2-acetoxybenzoic add, fumaric acid, toluenesulfonic acid, methanesulfonic add, ethane disulfonic add and oxalic acid, and inorganic acids such as hydrochloric acid, hydrobromic acid, sulphuric acid, sulfamic acid, phosphoric acid and nitric acid. Examples of suitable bases include inorganic bases such as sodium bicarbonate, sodium hydroxide and ammonium hydroxide. Examples of suitable plasticizers include triethyl citrate, glyceryl triacetate, tributyl citrate, diethyl phthalate, acetyl tributyl citrate, dibutyl phthalate and dibutyl sebacate. Examples of suitable glidants include talc, glyceryl monostearate, oleic acid, medium chain triglycerides and colloidal silicon dioxide. Examples of suitable surfactants include sodium dodecyl sulfate, polysorbate 80 and sorbitan monooleate.

In order to improve the adherence of the coating layer onto the cholestyramine pellets, or in order to minimize the interaction between the coating layer and the cholestyramine in the pellets, a barrier coating may optionally be present as an additional layer between the pellets and the coating layer. A barrier coating may also be present when two different coating layers should be kept physically separated from each other. A particularly suitable material for the barrier coating is hydroxypropyl methylcellulose (HPMC).

A thin layer of a non-sticking agent may ultimately be applied to the coated pellets. This outer layer prevents the coated pellets from sticking together, e.g. during storage. Examples of suitable non-sticking agents include fumed silica, talc and magnesium stearate.

Together, the coating layers substantially prevent release of the cholestyramine from the pellets until they have reached the large intestine. Additionally, because of the properties of the polymer in the diffusion-controlled inner coating, the cholestyramine is made available to the large intestine only slowly and during a period of several hours. Preferably, there should be no exposure of the cholestyramine in the small intestine, whereas the exposure should be quick once the multiparticulates have passed the ileocecal valve. In one embodiment, less than about 30% of the cholestyramine is released in the small intestine, such as less than about 20%, such as less than about 10%. In a more preferred embodiment, less than about 5% of the cholestyramine is released in the small intestine. In another embodiment, more than about 70% of the cholestyramine is released in the colon, such as more than about 80%, such as more than about 90%. In a more preferred embodiment, more than about 95% of the cholestyramine is released in the colon.

The coating layers add further weight and volume to the pellets. The smaller the size of the pellets, the larger is the impact of the coating on the volume of the final formulation. However, for reasons of patient compliance, it is desirable that the total volume of the formulation is kept as low as possible. The coating layers should therefore be as thin as possible. Preferably, the amount of coating in the final formulation (on dry weight basis) is less than about 40% w/w, and more preferably less than about 35% w/w.

The cholestyramine content of the pellets should be as high as possible. The uncoated pellets therefore preferably contain at least about 70% w/w cholestyramine, more preferably at least about 75% w/w cholestyramine, more preferably at least about 80% w/w cholestyramine, even more preferably at least about 85% w/w cholestyramine and most preferably at least about 90% w/w cholestyramine. The cholestyramine content of the final formulation (on dry weight basis) is preferably at least about 50% w/w, and more preferably at least about 55% w/w.

The size of the pellets is initially governed by the diameter of the screen used in the extrusion step. After the extrusion and spheronization steps, the pellets may be sieved to obtain a pellet fraction with a narrow size distribution. The diameter of the uncoated cholestyramine pellets is preferably from about 500 μm to about 3000 μm, more preferably from about 6000 μm to about 2000 μm and even more preferably from about 700 to about 1600 μm. In a most preferred embodiment, the diameter of the pellets is from about 700 to about 1000 μm, or from about 1000 to about 1400 μm.

The cholestyramine pellets may be prepared in a process comprising the steps of:
i) mixing the dry ingredients;
ii) adding water and the acrylate copolymer, to obtain a wet mass;
iii) extruding the wet mass;
iv) spheronizing the extrudate; and
v) drying the obtained pellets.

The dried pellets may thereafter be sieved in order to obtain pellets of uniform size.

The dry ingredients in step i) comprise cholestyramine, and may further comprise one or more of a vinylpyrrolidone-based polymer and microcrystalline cellulose.

Because of its physical nature, cholestyramine powder is able to absorb large amounts of water, which results in considerable swelling of the material. In order to prepare a wet mass from dry cholestyramine, it is therefore necessary to add more water than normally would be used for preparing a wet mass from dry ingredients. Preferably, water is added to the mix of dry ingredients in an amount of at least about 1.5 times the amount of cholestyramine (w/w), or in an amount of at least about 1.75 times the amount of cholestyramine (w/w), or in an amount of at least about 2 times the amount of cholestyramine (w/w). In some embodiments, the water:dry blend ratio is between about 1.5:1 and about 1.9:1, such as between about 1.6:1 and about 1.8:1. In some embodiments, the water:dry blend ratio is about 1.5:1. In some embodiments, the water:dry blend ratio is about 1.6:1. In some embodiments, the water:dry blend ratio is about 1.7:1. In some embodiments, the water:dry blend ratio is about 1.8:1. In some embodiments, the water:dry blend ratio is about 1.9:1.

In some embodiments, the dry blend:water ratio is between about 1.5 and about 1.9, such as between about 1.6 and about 1.8. In some embodiments, the dry blend:water ratio is about 1.5. In some embodiments, the dry blend:water ratio is about 1.6. In some embodiments, the dry blend:water ratio is about 1.7. In some embodiments, the dry blend:water ratio is about 1.8. In some embodiments, the dry blend:water ratio is about 1.9.

The coating may be applied onto the cholestyramine pellets by methods known in the art, such as by film coating involving perforated pans and fluidized beds.

The oral formulation described herein may be administered to a patient in different forms, depending on factors such as the age and general physical condition of the patient. For example, the formulation may be administered in the form of one or more capsules wherein the coated pellets are contained. Such capsules conventionally comprise a degradable material, such as gelatin, hydroxypropyl methylcellulose (HPMC), pullulan or starch, which easily disintegrates under the acidic conditions in the stomach. The coated pellets are thereby quickly released into the stomach. Thus, in one aspect, the invention relates to a capsule comprising the oral formulation disclosed herein.

Alternatively, the coated pellets may be administered as a sprinkle formulation, the contents of which can be dispersed in liquid or soft food. Such a formulation does not require the swallowing of larger capsules and is therefore particularly useful for infants and small children as well as for older adults. Thus, in another aspect, the invention relates to a sprinkle formulation comprising the oral formulation disclosed herein. In such a formulation, the coated pellets may be contained within a capsule, sachet or stick pack.

The oral formulation disclosed herein provides several advantages over other formulations. The small coated pellets (multiparticulates) according to the present invention are able to easily pass the gastrointestinal tract. This eliminates the risk that the formulation is temporarily held up in the gastrointestinal tract, such as at the stomach or at the ileocecal valve, as is sometimes encountered with monolithic formulations (such as tablets or capsules that do not disintegrate in the stomach). Furthermore, the cholestyramine is made available to the intestinal content only when the diffusion-controlled inner coating starts being degraded in the lower gastrointestinal tract, in particular the colon. The contents of the stomach and the small intestine are therefore effectively protected from the cholestyramine, which is a major improvement over formulations that directly release the cholestyramine in the stomach or the small intestine.

The low solubility of cholestyramine in aqueous environment prevents the release of cholestyramine from the formulation to be measured directly. The availability of the cholestyramine to the intestinal content over time and at different pH values can instead be determined in vitro, such as by measuring the sequestering capacity of the formulation under simulated conditions for the gastrointestinal tract. Such a method involves measuring the decreasing amount of free bile acid (i.e., the compound to be sequestered) in a liquid medium representative of the gastrointestinal tract, as described in the experimental section. See also the Official Monograph for cholestyramine resin (USP 40, page 3404).

In some embodiments, the sequestering capacities of a cholestyramine formulation is determined using the Simulator of the Human Intestinal Microbial Ecosystem (SHIME®) as developed by ProDigest (Ghent, Belgium). As described in more detail in the experimental section, this model enables the in vitro evaluation of the bile acid binding capacity of cholestyramine formulations under physiological conditions representative of a fasted stomach, small intestine and proximal colon. Bile acids such as cholic acid (CA), chenodeoxycholic acid (CDCA) and deoxycholic acid (DCA), or a mixture of two or more of these bile salts, may be used in such studies. A 40:40:20 (w/w) mixture of CA, CDCA and DCA is preferably used as a representative mixture of human bile salts. Experiments on cholestyramine formulations may be run in parallel with a control experiment to which no cholestyramine is added, in order to monitor the degradation of the bile salts under the conditions used in the assay. For each such experiment, samples are taken at selected time intervals and the concentrations of the bile acids in the samples are determined, e.g. by means of HPLC. From these data, the percentage of remaining bile acids in each studied sample may be calculated as the ratio of the value of the studied sample to the value of the control sample at the corresponding incubation time:

$$\% remaining bile acid = \frac{concentration of BA in sample}{concentration of BA in control sample} \times 100$$

A plot of the percentage of remaining bile acids against time will show the decrease of bile acids, i.e. the sequestration of bile acids by the cholestyramine formulations, during small intestinal and colonic incubation.

In another aspect, the invention relates to an oral formulation, comprising:
a) a plurality of pellets, each pellet comprising cholestyramine; and
b) a coating surrounding each pellet, wherein the coating is capable of targeting release of the cholestyramine in the colon;

wherein the oral formulation herein exhibits less than about about 30% sequestration of one or more of cholic acid, chenodeoxycholic acid, and deoxycholic acid after about 2 hours in small intestinal incubations as measured in the Simulator of the Human Intestinal Microbial Ecosystem (SHIME) model.

In some embodiments, the oral formulation exhibits less than about 25% sequestration of one or more of cholic acid, chenodeoxycholic acid, and deoxycholic acid after about 2 hours in small intestinal incubations as measured in the Simulator of the Human Intestinal Microbial Ecosystem (SHIME) model. In other embodiments, the oral formulation exhibits less than about 20% sequestration of one or more of cholic acid, chenodeoxycholic acid, and deoxycholic acid after about 2 hours in small intestinal incubations as measured in the Simulator of the Human Intestinal Microbial Ecosystem (SHIME) model. In yet other embodiments, the oral formulation exhibits less than about 15% sequestration of one or more of cholic acid, chenodeoxycholic acid, and deoxycholic acid after about 2 hours in small intestinal incubations as measured in the Simulator of the Human Intestinal Microbial Ecosystem (SHIME) model.

In another aspect, the invention relates to the formulation disclosed herein for use in the treatment or prevention of bile acid malabsorption.

The invention also relates to the use of the formulation disclosed herein in the manufacture of a medicament for the treatment or prevention of bile acid malabsorption. The invention further relates to a method for the treatment or prevention of bile acid malabsorption comprising administering to a mammal in need of such treatment or prevention a therapeutically effective amount of the formulation disclosed herein.

Bile acid malabsorption may be divided into three different types, dependent on the cause of the failure of the distal ileum to absorb bile acids. Type 1 BAM is the result of (terminal) ileal disease (such as Crohn's disease) or (terminal) ileal resection or bypass. Type 2 BAM is often referred to as idiopathic bile acid malabsorption or primary bile acid diarrhoea (BAD) and is believed to be the result of an overproduction of bile acids or caused by a defective feedback inhibition of hepatic bile acid synthesis. This feedback regulation is mediated by the ileal hormone fibroblast growth factor 19 (FGF19) in man. Finally, type 3 BAM may be the result of cholecystectomy, vagotomy, small intestinal bacterial overgrowth (SIBO), coeliac disease, pancreatic insufficiency (chronic pancreatitis, cystic fibrosis), pancreatic transplant, radiation enteritis, collagenous colitis, microscopic colitis, lymphocytic colitis, ulcerative colitis or irritable bowel syndrome (i.e., diarrhoea-predominant irritable bowel syndrome (IBS-D)).

The formulation may also be used in combination with an Ileal Bile Acid Absorption (IBAT) inhibitor. Treatment with IBAT inhibitors, such as in the treatment of liver diseases, disorders of fatty acid metabolism or glucose utilization disorders, may result in increased levels of bile acids and/or influence the reabsorption of bile acids by the small intestine, leading to high concentrations of bile acid in the large intestine and thus causing diarrhoea. This side effect of the treatment with IBAT inhibitors may be treated or prevented by treatment with the formulation as disclosed herein. The formulation and the IBAT inhibitor may be administered simultaneously, sequentially or separately.

Thus, in another aspect, the invention relates to the formulation disclosed herein, for use in the treatment or prevention of diarrhoea upon oral administration of an IBAT inhibitor.

The invention also relates to the use of the formulation disclosed herein in the manufacture of a medicament for the treatment or prevention of diarrhoea upon oral administration of an IBAT inhibitor. The invention further relates to a method for the treatment or prevention of diarrhoea upon oral administration of an IBAT inhibitor, comprising administering to a mammal in need of such treatment or prevention therapeutically effective amounts of an IBAT inhibitor and of the formulation disclosed herein.

In a preferred embodiment, the invention relates to the formulation disclosed herein, for use in the treatment or prevention of bile acid diarrhoea upon treatment of a liver disease, such as a cholestatic liver disease, comprising oral administration of an IBAT inhibitor. In particular, the invention relates to the formulation disclosed herein for use in the treatment or prevention of diarrhoea upon treatment of Alagilles syndrome (ALGS), progressive familial intrahepatic cholestasis (PFIC), primary biliary cirrhosis (PBC), primary sclerosing cholangitis (PSC), autoimmune hepatitis, cholestatic pruritus, non-alcoholic fatty liver disease (NAFLD) or non-alcoholic steatohepatitis (NASH) comprising oral administration of an IBAT inhibitor.

In another embodiment, the invention relates to a method for the treatment or prevention of bile acid diarrhoea upon treatment of a liver disease comprising oral administration of an IBAT inhibitor, comprising administering to a mammal in need of such treatment or prevention a therapeutically effective amount of the formulation disclosed herein. In particular, the invention relates to such a method for the treatment or prevention of diarrhoea wherein the liver disease is Alagilles syndrome (ALGS), progressive familial intrahepatic cholestasis (PFIC), primary biliary cirrhosis (PBC), primary sclerosing cholangitis (PSC), biliary atresia, autoimmune hepatitis, cholestatic pruritus, non-alcoholic fatty liver disease (NAFLD) or non-alcoholic steatohepatitis (NASH).

A liver disease as defined herein is any disease in the liver and in organs connected therewith, such as the pancreas, portal vein, the liver parenchyma, the intrahepatic biliary tree, the extrahepatic biliary tree, and the gall bladder. In some embodiments, a liver disease is a bile acid-dependent liver disease. In some embodiments, a liver disease involves elevated levels of bile acids in the serum and/or in the liver. In some embodiments, a liver disease is a cholestatic liver disease. Liver diseases and disorders include, but are not limited to an inherited metabolic disorder of the liver; inborn errors of bile acid synthesis; congenital bile duct anomalies; biliary atresia; post-Kasai biliary atresia; post-liver transplantation biliary atresia; neonatal hepatitis; neonatal cholestasis; hereditary forms of cholestasis; cerebrotendinous xanthomatosis; a secondary defect of BA synthesis; Zellweger's syndrome; cystic fibrosis-associated liver disease; alpha1-antitrypsin deficiency; Alagilles syndrome (ALGS); Byler syndrome; a primary defect of bile acid (BA) synthesis; progressive familial intrahepatic cholestasis (PFIC) including PFIC-1, PFIC-2, PFIC-3 and non-specified PFIC, post-biliary diversion PFIC and post-liver transplant PFIC; benign recurrent intrahepatic cholestasis (BRIC) including BRIC1, BRIC2 and non-specified BRIC, post-biliary diversion BRIC and post-liver transplant BRIC; autoimmune hepatitis; primary biliary cirrhosis (PBC); liver fibrosis; non-alcoholic fatty liver disease (NAFLD); non-alcoholic steatohepatitis (NASH); portal hypertension; cholestasis; Down syndrome cholestasis; drug-induced cholestasis; intrahepatic cholestasis of pregnancy (jaundice during pregnancy); intrahepatic cholestasis; extrahepatic cholestasis; parenteral nutrition associated cholestasis (PNAC); low phospholipid-associated cholestasis; lymphedema cholestasis syndrome 1 (LSC1); primary sclerosing cholangitis (PSC); immunoglobulin G4 associated cholangitis; primary biliary cholangitis; cholelithiasis (gall stones); biliary lithiasis; choledocholithiasis; gallstone pancreatitis; Caroli disease; malignancy of bile ducts; malignancy causing obstruction of the biliary tree; biliary strictures; AIDS cholangiopathy; ischemic cholangiopathy; pruritus due to cholestasis or jaundice; pancreatitis; chronic autoimmune liver disease leading to progressive cholestasis; hepatic steatosis; alcoholic hepatitis; acute fatty liver; fatty liver of pregnancy; drug-induced hepatitis; iron overload disorders; congenital bile acid synthesis defect type 1 (BAS type 1); drug-induced liver injury (DILI); hepatic fibrosis; congenital hepatic fibrosis; hepatic cirrhosis; Langerhans cell histiocytosis (LCH); neonatal ichthyosis sclerosing cholangitis (NISCH); erythropoietic protoporphyria (EPP); idiopathic adulthood ductopenia (IAD); idiopathic neonatal hepatitis (INH); non syndromic paucity of interlobular bile ducts (NS PILBD); North American Indian childhood cirrhosis (NAIC); hepatic sarcoidosis; amyloidosis; necrotizing enterocolitis; serum bile acid-caused toxicities, including cardiac rhythm disturbances (e.g., atrial fibrillation) in setting of abnormal serum bile acid profile, cardiomyopathy associated with liver cirrhosis (“cholecardia”), and skeletal muscle wasting associated with cholestatic liver disease; viral hepatitis (including hepatitis A, hepatitis B, hepatitis C, hepatitis D and hepatitis E); hepatocellular carcinoma (hepatoma); cholangiocarcinoma; bile acid-related gastrointestinal cancers; and cholestasis caused by tumours and neoplasms of the liver, of the biliary tract and of the pancreas.

Disorders of fatty acid metabolism and glucose utilization disorders include, but are not limited to, hypercholesterolemia, dyslipidemia, metabolic syndrome, obesity, disorders of fatty acid metabolism, glucose utilization disorders, disorders in which insulin resistance is involved, and type 1 and type 2 diabetes mellitus.

IBAT inhibitors are often referred to by different names. As used herein, the term “IBAT inhibitors” should be understood as also encompassing compounds known in the literature as Apical Sodium-dependent Bile Acid Transporter Inhibitors (ASBTI's), bile acid transporter (BAT) inhibitors, ileal sodium/bile acid cotransporter system inhibitors, apical sodium-bile acid cotransporter inhibitors, ileal sodium-dependent bile acid transport inhibitors, bile acid reabsorption inhibitors (BARI's), and sodium bile acid transporter (SBAT) inhibitors.

IBAT inhibitors that can be used in combination with the bile acid sequestrant formulation disclosed herein include, but are not limited to, benzothiazepines, benzothiepines, 1,4-benzothiazepines, 1,5-benzothiazepines and 1,2,5-benzothiadiazepines.

Suitable examples of IBAT inhibitors that can be used in combination with the bile acid sequestrant formulation disclosed herein include, but are not limited to, the compounds disclosed in WO 93/16055, WO 94/18183, WO 94/18184, WO 96/05188, WO 96/08484, WO 96/16051, WO 97/33882, WO 98/03818, WO 98/07449, WO 98/40375, WO 99/35135, WO 99/64409, WO 99/64410, WO 00/47568, WO00/61568, WO 00/38725, WO 00/38726, WO 00/38727, WO 00/38728, WO 00/38729, WO 01/68096, WO 02/32428, WO 03/061663, WO 2004/006899, WO 2007/009655, WO 2007/009656, DE 19825804, EP 864582, EP 489423, EP 549967, EP 573848, EP 624593, EP 624594, EP 624595, EP 624596, EP 0864582, EP 1173205 and EP 1535913; all of which are hereby incorporated by reference in their entireties.

Particularly suitable IBAT inhibitors are those disclosed in WO 01/66533, WO 02/50051, WO 03/022286, WO 03/020710, WO 03/022825, WO 03/022830, WO 03/091232, WO 03/106482, WO 2004/076430 and PCT/EP2019/064602, all of which are hereby incorporated by reference in their entireties, and especially the compounds selected from the group consisting of:

1,1-dioxo-3,3-dibutyl-5-phenyl-7-methylthio-8-(N-{(R)-α-[N-(carboxymethyl)carbamoyl]-benzyl}carbamoylmethoxy)-2,3,4,5-tetrahydro-1,2,5-benzothiadiazepine;

1,1-dioxo-3,3-dibutyl-5-phenyl-7-methylthio-8-(N-{(R)-α-[N′-((S)-1-carboxyethyl)carbamoyl]-benzyl}carbamoylmethoxy)-2,3,4,5-tetrahydro-1,5-benzothiazepine;

1,1-dioxo-3,3-dibutyl-5-phenyl-7-methylthio-8-(N-{(R)-α-[N-((S)-1-carboxypropyl)-carbamoyl]benzyl}carbamoylmethoxy)-2,3,4,5-tetrahydro-1,2,5-benzothiadiazepine;

1,1-dioxo-3,3-dibutyl-5-phenyl-7-methylthio-8-(N-{(R)-α-[N-((R)-1-carboxy-2-methylthioethyl)-carbamoyl]benzyl}carbamoylmethoxy)-2,3,4,5-tetrahydro-1,2,5-benzothiadiazepine;

1,1-dioxo-3,3-dibutyl-5-phenyl-7-methylthio-8-(N-{(R)-α-[N-((S)-1-carboxypropyl)carbamoyl]-4-hydroxybenzyl}carbamoylmethoxy)-2,3,4,5-tetrahydro-1,2,5-benzothiadiazepine;

1,1-dioxo-3,3-dibutyl-5-phenyl-7-methylthio-8-(N-{(R)-α-[N-((R)-1-carboxy-2-methylthio-ethyl)carbamoyl]-4-hydroxybenzyl}carbamoylmethoxy)-2,3,4,5-tetrahydro-1,2,5-benzothiadiazepine;

1,1-dioxo-3,3-dibutyl-5-phenyl-7-methylthio-8-(N-{(R)-α-[N-((S)-1-carboxy-2-methylpropyl)-carbamoyl]benzyl}carbamoylmethoxy)-2,3,4,5-tetrahydro-1,2,5-benzothiadiazepine;

1,1-dioxo-3,3-dibutyl-5-phenyl-7-methylthio-8-(N-{(R)-α-[N-((S)-1-carboxy-2-(R)-hydroxypropyl)carbamoyl]-4-hydroxybenzyl}carbamoylmethoxy)-2,3,4,5-tetrahydro-1,2,5-benzothiadiazepine;

1,1-dioxo-3,3-dibutyl-5-phenyl-7-methylthio-8-(N-{(R)-α-[N-((S)-1-carboxybutyl)carbamoyl]-4-hydroxybenzyl}carbamoylmethoxy)-2,3,4,5-tetrahydro-1,2,5-benzothiadiazepine;

1,1-dioxo-3,3-dibutyl-5-phenyl-7-methylthio-8-(N-{(R)-α-[N-((S)-1-carboxyethyl)carbamoyl]-benzyl}carbamoylmethoxy)-2,3,4,5-tetrahydro-1,2,5-benzothiadiazepine;

1, 1-dioxo-3, 3-dibutyl-5-phenyl-7-methylthio-8-(N-{(R)-α-[N′((S)-1-carboxypropyl)carbamoyl]-4-hydroxybenzyl}carbamoylmethoxy)-2,3,4,5-tetrahydro-1,5-benzothiazepine;

1,1-dioxo-3,3-dibutyl-5-phenyl-7-methylthio-8-(N-{(R)-α-[N-((S)-1-carboxyethyl)carbamoyl]-4-hydroxybenzyl}carbamoylmethoxy)-2,3,4,5-tetrahydro-1,2,5-benzothiadiazepine;

1,1-dioxo-3,3-dibutyl-5-phenyl-7-methylthio-8-(N-{(R)-α-[N-((S)-1-carboxy-2-methylpropyl)-carbamoyl]-4-hydroxybenzyl}carbamoylmethoxy)-2,3,4,5-tetrahydro-1,2,5-benzothiadiazepine; and

1,1-dioxo-3,3-dibutyl-5-phenyl-7-methylthio-8-(N-{(R)-1′-phenyl-1′-[N′-(carboxymethyl)carbamoyl] methyl}carbamoylmethoxy)-2,3,4,5-tetrahydro-1,5-benzothiazepine;

or a pharmaceutically acceptable salt thereof.

Other particularly suitable IBAT inhibitors are those disclosed in WO99/32478, WO00/01687, WO01/68637, WO03/022804, WO 2008/058628 and WO 2008/058630, all of which are hereby incorporated by reference in their entireties, and especially the compounds selected from the group consisting of:

1-[4-[4-[(4R,5R)-3,3-dibutyl-7-(dimethylamino)-2,3,4,5-tetrahydro-4-hydroxy-1,1-dioxido-1-benzothiepin-5-yl]phenoxy]butyl]4-aza-1-azoniabicyclo[2.2.2]octane methanesulfonate;

1-[[4-[[4-[3,3-dibutyl-7-(dimethylamino)-2,3,4,5-tetrahydro-4-hydroxy-1,1-dioxido-1-benzothiepin-5-yl]phenoxy]methyl]phenyl]methyl]-4-aza-1-azoniazabicyclo[2.2.2]octane chloride;

1-[[5-[[3-[(3S,4R,5R)-3-butyl-7-(dimethylamino)-3-ethyl-2,3,4,5-tetrahydro-4-hydroxy-1,1-dioxido-1-benzothiepin-5-yl]phenyl]amino]-5-oxopentyl]amino]-1-deoxy-D-glucitol; and

potassium ((2R,3R,4S,5R,6R)-4-benzyloxy-6-{3-[3-((3S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydro-pyran-2-ylmethyl)sulphate, ethanolate, hydrate.

An effective amount of the cholestyramine formulation according to the invention can be any amount containing more than or equal to about 100 mg of cholestyramine, such as more than or equal to about 250 mg, about 500 mg, about 750 mg, about 1000 mg, about 1250 mg, about 1500 mg, about 1750 mg or about 2000 mg of cholestyramine. For example, the effective amount of cholestyramine can be between about 100 mg and about 5000 mg, such as between about 250 mg and about 2500 mg, between about 250 mg and about 2000 mg, between about 500 mg and about 2500 mg, between about 500 mg and about 2000 mg, or between about 750 mg and about 2000 mg.

A unit dose of the cholestyramine formulation according to the invention may comprise from about 200 to about 300 mg of cholestyramine, such as from about 220 to about 280 mg of cholestyramine, such as from about 240 to about 260 mg of cholestyramine. A unit dose preferably comprises about 250 mg of cholestyramine. The daily dose can be administered as a single dose or divided into one, two, three or more unit doses.

The frequency of administration of the formulation as disclosed herein can be any frequency that reduces the bile acid malabsorption condition without causing any significant adverse effects or toxicity to the patient. The frequency of administration can vary from once or twice a week to several times a day, such as once a day or twice a day. The frequency of administration can furthermore remain constant or be variable during the duration of the treatment.

Several factors can influence the frequency of administration and the effective amount of the formulation that should be used for a particular application, such as the severity of the condition being treated, the duration of the treatment, as well as the age, weight, sex, diet and general medical condition of the patient being treated.

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

The invention is further illustrated by means of the following examples, which do not limit the invention in any respect. All cited documents and references are incorporated herein by reference.

Abbreviations

HPLC High Performance Liquid Chromatography

PTFE Polytetrafluoroethylene

RH Relative humidity

rpm revolutions per minute

UHPLC Ultra High Performance Liquid Chromatography

UV-Vis Ultraviolet-visible spectroscopy

### EXAMPLES

### Example 1

Extrusion/Spheronization Experiments

All experiments were performed on a 100-200 g scale. The dry ingredients (cholestyramine, the vinylpyrrolidone-based polymer and/or microcrystalline cellulose) were mixed in the amounts indicated below. Water was added in portions of 50-100 gram with 3 minutes of mixing between each addition. When an acrylate copolymer was included in the experiment, it was added as a 2% w/w dispersion in water (20 g acrylate copolymer (aqueous dispersion 30%) added up to 300 g water). A final portion of pure water was added, if necessary. In each experiment, the total amount of liquid added was between 1.7 and 2.3 times the amount of solid material (w/w).

The wet mass was transferred to an extruder equipped with a 1.5 mm screen, operated at 25 rpm (revolutions per minute) and the extrudate was collected on a stainless steel tray. Approximately 100 g of the extrudate was run in the spheronizer for 1 minute at a speed of 730 rpm. The spheronized material was then transferred to stainless steel trays, placed in a drying oven and dried for 16 hours at 50° C. The yield was calculated as the fraction of pellets that pass through a 1.6 mm sieve but are retained on a 1.0 mm sieve.

Friability testing was performed using the equipment and procedure described in European Pharmacopoeia 8.0, test 2.9.7. The pellets were sieved on a 500 μm sieve to remove any loose dust before weighing.

The results using copovidone and Eudragit® RL 30 D are shown in Table 1, and the results using povidone and other Eudragit® copolymers are shown in Table 2.

TABLE 1

| | Amount (% w/w) | | | | | |
|---|---|---|---|---|---|---|
| Entry | Cholestyramine | Copovidone | MCC | Eudragit ® RL 30 D | Yield (%) | Friability (%) |
| 1 | 100 | 0 | 0 | 0 | * | * |
| 2 | 90 | 0 | 10 | 0 | * | * |
| 3 | 70 | 0 | 30 | 0 | 39 | 1.6 |
| 4 | 70 | 6 | 24 | 0 | * | * |
| 5 | 70 | 0 | 26 | 4 | * | * |
| 6 | 70 | 6 | 20 | 4 | 85 | 0.1 |
| 7 | 80 | 3 | 15 | 2 | * | * |
| 8 | 85 | 7.5 | 4.5 | 3 | 92 | 0.6 |
| 9 | 90 | 6 | 4 | 0 | * | * |
| 10 | 90 | 0 | 6 | 4 | * | * |
| 11 | 90 | 0 | 0 | 10 | * | * |
| 12 | 90 | 6 | 0 | 4 | 85 | 1.4 |
| 13 | 90 | 10 | 0 | 0 | 87 | 1.2 |
| 14 | 91 | 9 | 0 | 0 | 82 | 0.5 |
| 15 | 92 | 8 | 0 | 0 | 83 | 1.5 |
| 16 | 93 | 7 | 0 | 0 | 78 | 1.0 |
| 17 | 94 | 6 | 0 | 0 | * | * |
| 18 | 91 | 6 | 0 | 3 | 84 | 0.3 |
| 19 | 92 | 6 | 0 | 2 | 82 | 1.6 |
| 20 | 93 | 6 | 0 | 1 | * | * |
| 21 | 85 | 6 | 8 | 1 | 81 | 3.5 |
| 22 | 80 | 6 | 13 | 1 | 85 | 0.8 |
| 23 | 92 | 5 | 0 | 3 | 70 | 2.0 |
| 24 | 93 | 5 | 0 | 2 | * | * |
| 25 | 85 | 5 | 8 | 2 | 54 | 7.1 |
| 26 | 80 | 5 | 13 | 2 | 73 | 9.1 |

* = extrusion followed by spheronization did not lead to pellets.

TABLE 2

| | Amount (% w/w) | | | | | |
|---|---|---|---|---|---|---|
| Entry | Cholestyramine | Povidone | MCC | Eudragit ® | Yield (%) | Friability (%) |
| 1 | 85 | 7.5 | 4.5 | 3% w/w FS 30 D | 79 | 0.2 |
| 2 | 85 | 7.5 | 4.5 | 3% w/w L 30 D-55 | 24 | 0.8 |
| 3 | 85 | 7.5 | 4.5 | 3% w/w NE 30 D | 88 | 0.5 |
| 4 | 85 | 7.5 | 4.5 | 3% w/w NM 30 D | 96 | 0.9 |
| 5 | 85 | 7.5 | 4.5 | 3% w/w RS 30 D | 82 | 0.8 |

### Example 2

Preparation of Pellets (200 g Scale; 85% (w/w) Cholestyramine)

Pellets with a composition according to Table 1, entry 8, were manufactured at a batch size of 200 g in the extrusion step and 100 g in the spheronization step. 170 g cholestyramine, 15 g copovidone and 9 g microcrystalline cellulose were charged into a planetary mixer. The mixer was operated at intermediate speed and the liquid was slowly added in portions with mixing between each addition. First 300 g water with 20 g Eudragit® RL 30 D (30% dry weight) was added in three equal portions, with mixing for 3 minutes between each addition. Finally 40 g pure water was added and mixing was performed for additionally 30 seconds. The wet mass was then transferred to the extruder. The extruder was equipped with a 1.5 mm screen, operated at 25 rpm and the extrudate was collected on a stainless steel tray. Approximately 100 g of the extrudate was run in the spheronizer for 1 minute at a speed of 730 rpm. The spheronized material was then transferred to stainless steel trays, placed in a drying oven and dried for 16 hours at 50° C. The dried pellets were sieved and the fraction between 1 mm and 1.4 mm was collected.

### Example 3

Preparation of Pellets (5 kg Scale; 80% (w/w) Cholestyramine)

Cholestyramine (Purolite A430MR; 4000 g), copovidone (Kollidone VA64 fine; 375 g) and microcrystalline cellulose (Avicel PH 101; 225 g) were charged into a Hobart Low Shear granulator bowl and the dry powder was mixed at intermediate speed for 1 minute. A stirred suspension of Eudragit® RL 30 D (1333 g; 30% dry weight) in purified water (about 6.5 L) was sprayed into the granulator at a speed of 1625 g/min. After addition of the suspension, the mass was mixed for an additional 4 minutes. Purified water (about 1 L) was then sprayed into the granulator at a speed of 1050 g/min, and the mass was mixed for 1 additional minute. In total, the dry blend:water ratio was about 1:1.7.

The wet mass was transferred to the extruder (MG-55, LCI Corporation), equipped with a 1.0 mm dome screen and operating at 50 rpm. The extrudate was then transferred to a QJ-400 spheronizer equipped with a 2 mm friction plate. The extrudate was spheronized in portions of 1 kg for 15 minutes at a speed of 500 rpm. The spheronized material was then dried in a Huettlin Unilab fluid bed dryer for 24 hours at 55° C. The dried pellets were sieved through 18 and 25 mesh screens, and the material retained on 25 mesh screens (corresponding to pellets with a size between 0.7 and 1.0 mm) was collected.

### Example 4

Formulations A-C for pH- and Diffusion-Controlled Release

The cholestyramine pellets of Example 2 were formulated with a colon release coating comprising an diffusion controlled inner coating based on poly(ethyl acrylate-co-methyl methacrylate-co-trimethylammonioethyl methacrylate chloride) and an enteric outer coating based on hydroxypropyl methylcellulose acetate succinate.

Three formulations were prepared with different amounts of poly(ethyl acrylate-co-methyl methacrylate-co-trimethylammonioethyl methacrylate chloride) in the inner coating, as follows:

Formulation A: 100% Eudragit® RL 30 D

Formulation B: 50% Eudragit® RL 30 D+50% Eudragit® RS 30 D

Formulation C: 100% Eudragit® RS 30 D

The pellets composition for a unit dose comprising 250 mg cholestyramine is shown below.

| | Amount |
|---|---|
| Ingredient | (mg/dose) |
| Cholestyramine | 250 |
| Copovidone (Kollidon ® VA64 Fine) | 22.1 |
| Microcrystalline cellulose (Avicel ® PH102) | 13.2 |
| Poly(ethyl acrylate-co-methyl methacrylate-co- | 8.8 |
| trimethylammonioethyl methacrylate chloride) | |
| 1:2:0.2 (Eudragit ® RL 30 D) | |
| Total | 294.1 |

Inner Coating

A glycerol monostearate (GMS) emulsion containing GMS, polysorbate 80 and triethyl citrate was prepared according to general instructions from Evonik. The emulsion was mixed with Eudragit RL30D/RS30D dispersion (30% w/w). The composition of the inner coating film, based on dry weight, is shown below. The concentration, based on dry weight of the applied dispersion, is 19.8% (w/w).

| Ingredient | Amount |
|---|---|
| Inner coating | (w/w) |
| ethyl acrylate-co-methyl methacrylate-co-trimethylammonioethyl | 90.4 |
| methacrylate chloride) 1:2:0.2 (Eudragit ® RL 30 D) or 1:2:0.1 | |
| (Eudragit ® RS 30 D) | |
| Triethyl citrate | 4.5 |
| Glycerol monostearate 45-55 (Kolliwax ® GMS II) | 3.6 |
| Polysorbate 80 (Tween ® 80) | 1.5 |

The coating layer was applied using a Hüttlin Kugelcoater HKC005; batch size 75 g. The coating process was performed with an air inlet temperature of 45° C., resulting in a product temperature of 27-29° C. Air flow was adjusted to achieve an appropriate fluidization of the pellets during the coating. The coating was applied to the pellets so as to obtain a weight gain of 10%. After the coating, the pellets were heat-treated at 40° C. for 24 hours.

Outer Coating

The enteric coating was prepared by mixing 7% w/w hypromellose acetate succinate, 2.45% w/w triethyl citrate, 2.1% w/w talc, 0.21% w/w sodium lauryl sulphate and 88.24% w/w water for 30 min with an overhead stirrer at low temperature, <15° C. The composition of the outer coating film, based on dry weight, is shown below. The coating liquid was kept below 15° C. during the coating process.

| Ingredient | Amount |
|---|---|
| Outer coating | (w/w) |
| Hypromellose acetate succinate (AQOAT AS HF) | 59.5 |
| Triethyl citrate | 20.8 |
| Talc, micronized | 17.9 |
| Sodium lauryl sulphate (Kolliphor ® SLS Fine) | 1.8 |

The coating layer was applied using a Hüttlin Kugelcoater HKC005; batch size 75 g. The coating process was performed with an air inlet temperature of 55° C., resulting in a product temperature of 32° C. Air flow was adjusted to achieve an appropriate fluidization of the pellets during the coating. The enteric coating was applied to the pellets so as to obtain a weight gain of 40% (based on the weight of the coated pellets after application of the inner coating). After the coating, the pellets were heat-treated at 40° C./75% RH for 48 hours.

The coated pellets may be encapsulated in capsules, e.g. hard gelatine capsules. Details for the final formulations (on dry weight basis) are shown below:
Dose weight: 452.9 mg
Cholestyramine: 250 mg (55%)
   Inner coating: 29.4 mg
   Outer coating: 129.4 mg
Total coating: 158.8 mg (35%)

### Example 5

Formulation D for pH- and Diffusion-Controlled Release

The cholestyramine pellets of Example 2 were formulated with a colon release coating comprising a diffusion controlled inner coating based on poly(ethyl acrylate-co-methyl methacrylate-co-trimethylammonioethyl methacrylate chloride), an enteric coating based on hydroxypropyl methylcellulose acetate succinate and finally coated with fumed silica to prevent sticking of the pellets during storage.

The pellets composition for a unit dose comprising 250 mg cholestyramine is shown below.

| | Amount |
|---|---|
| Ingredient | (mg/dose) |
| Cholestyramine | 250 |
| Copovidone (Kollidon ® VA64 Fine) | 22.1 |
| Microcrystalline cellulose (Avicel ® PH102) | 13.2 |
| Poly(ethyl acrylate-co-methyl methacrylate-co- | 8.8 |
| trimethylammonioethyl methacrylate chloride) | |
| 1:2:0.2 (Eudragit ® RL 30 D) | |
| Total | 294.1 |

Inner Coating

A glycerol monostearate (GMS) emulsion containing GMS, polysorbate 80 and triethyl citrate was prepared according to general instructions from Evonik. The emulsion was mixed with Eudragit RS30D dispersion (30% w/w). The composition of the inner coating film, based on dry weight, is shown below. The concentration, based on dry weight of the applied dispersion, is 20.0% (w/w).

| Ingredient | Amount |
|---|---|
| Inner coating | (w/w) |
| Poly(ethyl acrylate-co-methyl methacrylate-co- | 78.75 |
| trimethylammonioethyl methacrylate chloride) | |
| 1:2:0.1 (Eudragit ® RS 30 D) | |
| Triethyl citrate | 15.75 |
| Glycerol monostearate 45-55 (Kolliwax ® GMS II) | 3.95 |
| Polysorbate 80 (Tween ® 80) | 1.55 |

The coating solution was applied using a Vector FL-M-1 apparatus. The initial batch size was 500 g. The coating process was performed with an air inlet temperature of 41-43° C., resulting in a product temperature of 28-30° C. The air flow was adjusted to achieve an appropriate fluidization of the pellets during the coating. The coating was applied to the cholestyramine pellets so as to obtain a weight gain of 10%. The coated pellets were then heat-treated at 40° C. for 50 hours and 30 minutes.

Enteric Coating

The enteric coating was prepared by mixing 7% w/w hypromellose acetate succinate, 2.45% w/w triethyl citrate, 2.1% w/w talc, 0.21% w/w sodium lauryl sulphate and 88.24% w/w water for 30 minutes with an overhead stirrer at low temperature, <15° C. The composition of the outer coating film, based on dry weight, is shown below. The coating liquid was kept below 15° C. during the coating process.

| Ingredient | Amount |
|---|---|
| Outer coating | (w/w) |
| Hypromellose acetate succinate (AQOAT AS HF) | 59.5 |
| Triethyl citrate | 20.8 |
| Talc, micronized | 17.9 |
| Sodium lauryl sulphate (Kolliphor ® SLS Fine) | 1.8 |

The coating layer was applied using a Vector FL-M-1 apparatus. The coating process was performed with an air inlet temperature of 35-55° C., resulting in a product temperature of 28-32° C. Air flow was adjusted to achieve an appropriate fluidization of the pellets during the coating. The enteric coating was applied to the pellets so as to obtain a weight gain of 40% (based on the weight of the coated pellets after application of the inner coating).

Final Coating

Directly after the enteric coating, fumed silica was applied onto the coated pellets by spraying a 5% suspension of Aerosil® 200 in water onto the pellets. The coating was applied using the same equipment with an inlet temperature of 40-41° C., resulting in a product temperature of 30° C. The air flow was adjusted to achieve an appropriate fluidization of the pellets during the coating. The coating was applied to the cholestyramine pellets so as to obtain a weight gain of 1% (w/w). The coated pellets were finally in-process heat-treated at 60° C. for 30 minutes in the coating equipment.

The coated pellets may be encapsulated in capsules, e.g. hard gelatine capsules. Details for the final formulations (on dry weight basis) are shown below:
Dose weight: 457.4 mg
Cholestyramine: 250 mg (55%)
   Inner coating: 29.4 mg
   Enteric coating: 129.4 mg
Anti-sticking coating 4.5 mg
Total coating: 163.3 mg (36%)

### Example 6

Sequestration Assay

The sequestering capacities of formulations A, B and C were determined in a simplified assay, simulating the pH of the stomach and the small intestine. The sequestration was determined by measuring the decreasing amount of cholic acid in an aqueous solution. The USP Dissolution Apparatus 2 (paddle) Ph. Eur. 2.9.3 was used.

Sequestration at pH 5.5

An amount of formulation A, B or C corresponding to 250 mg cholestyramine was added to a vessel containing 500 mL of a buffered solution of cholic acid (0.192 mg/mL), pH 5.5 and the contents were stirred at 75 rpm for 6 hours. Samples of the solution were withdrawn at different time points and analysed for cholic acid by HPLC using a Thermo Hypersil Gold column, 50 mm×2.1 mm, particle size 1.9 μm; column temperature 60° C.; mobile phase 30:70 acetonitrile: phosphate buffer (pH 3.0); flow rate 0.75 mL/min. 5 replicate samples were analysed for each formulation and the average values were calculated.

Sequestration at pH 6.8 or 7.4

An amount of formulation A, B or C corresponding to 250 mg cholestyramine was added to a vessel containing 250 mL 0.1 M hydrochloric acid solution (pH 1) and the contents were stirred at 75 rpm for 2 hours. 250 mL of a solution of cholic acid in potassium hydroxide/potassium phosphate buffer solution was then added to the vessel, giving a buffered solution of cholic acid (0.192 mg/mL) with pH 6.8 or 7.4. After 1 minute of mixing, a first sample was removed. The pH was thereafter verified and if necessary adjusted to 6.8 or 7.4 by addition of the appropriate amount of 0.1 M potassium hydroxide solution. The solution was thereafter mixed for an additional 6 hours. Samples of the solution were withdrawn at different time points and analysed for cholic acid by HPLC using a Thermo Hypersil Gold column, 50 mm×2.1 mm, particle size 1.9 μm; column temperature 60° C.; mobile phase 30:70 acetonitrile: phosphate buffer (pH 3.0); flow rate 0.75 mL/min. 5 replicate samples were analysed for each formulation and the average values were calculated.

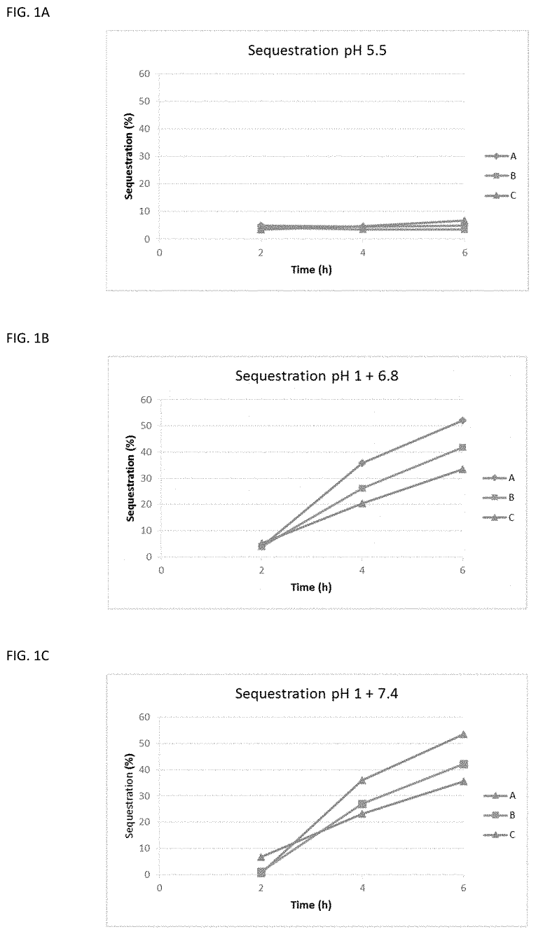

The sequestration profiles for formulations A-C are shown in FIG. 1. The pH of 5.5 is slightly lower than the pH normally observed in the duodenum, although it may occur in some patients and healthy persons. At this pH, sequestration is limited for all formulations (FIG. 1A). Sequestration at pH 6.8 is representative for the conditions in the ileum. At this pH, formulation A, B and C gave 52%, 42% and 34% sequestration, respectively, after 4 hours (FIG. 1B). At pH 7.4, formulation A, B and C gave 54%, 42% and 36% sequestration, respectively, after 4 hours (FIG. 1C). This pH is probably slightly higher than the pH normally observed in the distal ileum.

The coated pellets of formulations A, B and C showed no or only minor disintegration. Visual inspection of the pellets revealed that the coating was intact after stirring for 6 hours. In contrast, the uncoated pellets of Example 2, when stirred in a phosphate buffer (50 mM, pH 6.8) at 300 rpm (propeller stirrer), fully disintegrated within 1 minute and 25 seconds.

### Example 7

In Vitro Determination of the Sequestering Capacity of Formulations A-C Under Simulated Conditions for the Gastrointestinal Tract

The sequestering capacities of formulations A, B and C were studied in the Simulator of the Human Intestinal Microbial Ecosystem (SHIME®) as developed by ProDigest (Ghent, Belgium). The simulator was adapted to evaluate the sequestering capacity of binding bile salts under physiological conditions representative for fasted stomach, small intestine and proximal colon. The liquid media representative of the fasted stomach and small intestine have previously been described by Marzorati et al. (LWT-Food Sci. Technol. 2015, vol. 60, p. 544-551). The liquid medium for the proximal colon comprises a SHIME® matrix containing a stable microbial community representative for the human colon. A method for obtaining a stable microbial community of the human intestine is described by Possemiers et al. (FEMS Microbiol. Ecol. 2004, vol. 49, p. 495-507) and references therein. The sequestration was determined by measuring the decreasing amount of bile acids in an aqueous solution. A 40:40:20 (w/w) mixture of cholic acid (CA), chenodeoxycholic acid (CDCA) and deoxycholic acid (DCA) was used as a representative mixture of human bile salts (Carulli et al., Aliment. Pharmacol. Ther. 2000, vol. 14, issue supplement s2, p. 14-18).

A comparative experiment to which pure (unformulated) cholestyramine powder was added was also conducted. A control experiment to which no cholestyramine was added was conducted in order to monitor the degradation of the bile salts under the colonic conditions used in the assay.

Each experiment was performed in triplicate to account for biological variation.

Fasted Stomach

Amounts of formulations A, B and C corresponding to 91 mg of cholestyramine and the pure cholestyramine (91 mg) were dosed to 14 mL fasted stomach liquid medium (pH 1.8). The digests were incubated for 1 hour at 37° C.

Small Intestine

After one hour of stomach incubation, 5.6 mL pancreatic juice (pH 6.8) containing the defined 40:40:20 mixture of bile salts (46.7 mM) was added. The small intestine digests were incubated for 2 hours at 37° C. and samples were taken after 0, 60 and 120 minutes.

Proximal Colon

After two hours of small intestine incubation, 42 mL of a full SHIME matrix (pH 6.0) originated from the ascending colon of a SHIME system was added. The colon digests were incubated for 24 hours at 37° C. and samples were collected every hour for the first 6 hours and then at 19 h and at 24 h.

Sample Analysis

The concentration of free bile salts in the samples was assessed by means of HPLC. A calibration curve was used to calculate the concentrations of CA, CDCA and DCA in the samples. One mL of each sample was centrifuged for 2 min at 5000 g. 500 μL of the supernatant was mixed with 500 μL of an 80:20 (v:v) mixture of methanol and phosphate buffer, vigorously vortexed, filtered through a 0.2 μm PTFE filter and injected in a Hitachi Chromaster HPLC equipped with a UV-Vis detector. The three bile salts were separated by a reversed-phase C18 column (Hydro-RP, 4 μm, 80 Å, 250×4.6 mm, Synergi). The separation was performed under isocratic conditions at room temperature, using a 80:20 (v:v) mixture of methanol and phosphate buffer as the mobile phase. The analysis was performed at 0.7 mL/min during 23 minutes and the bile salts were detected at 210 nm. The injection volume was set at 20 μL for stomach and small intestine samples and 50 μL for colon samples.

The full SHIME® matrix that was used for the colonic incubations contains (degraded) bile salts originating from BD Difco™ Oxgall, a dehydrated fresh bile extract from bovine origin (Catalog Number 212820). Although the exact composition of this mixture is unknown, a higher quantity of free bile salts might be expected in the colon samples. The values of the background (i.e. blank sample where no mix of bile salts was added) were therefore subtracted from each sample in order to take into account the ‘baseline’ of free bile salts present in the total SHIME® matrix.

Tables 3, 4 and 5 below show the concentrations (mg/L) of CA, CDCA and DCA, respectively, that were measured in the samples collected during small intestinal (SI) and colonic incubation. The bottom row for each formulation indicates the percentage of remaining bile acids, calculated as the ratio of the value of each sample (pure cholestyramine or formulations A-C) to the value of the control sample at the corresponding incubation time.

TABLE 3
Concentrations of CA (mg/L) measured during incubation

| | SI incubation | | | Colonic incubation | | | | | | | | |
|---|---|---|---|---|---|---|---|---|---|---|---|---|
| | 0 h | 1 h | 2 h | 0 h | 1 h | 2 h | 3 h | 4 h | 5 h | 6 h | 19 h | 24 h |
| Control | 2410 | 2358 | 2360 | 604 | 598 | 564 | 539 | 534 | 492 | 470 | 392 | 349 |
| Cholestyramine | 2410 | 1081 | 989 | 161 | 80 | 70 | 65 | 31 | 15 | 0 | 0 | 0 |
| | 100% | 46% | 42% | 27% | 13% | 12% | 12% | 6% | 3% | 0% | 0% | 0% |
| Formulation A | 2410 | 2215 | 2018 | 466 | 377 | 342 | 298 | 234 | 169 | 134 | 0 | 0 |
| | 100% | 94% | 86% | 77% | 63% | 61% | 55% | 44% | 34% | 29% | 0% | 0% |
| Formulation B | 2410 | 2143 | 1882 | 425 | 367 | 307 | 245 | 192 | 149 | 91 | 0 | 0 |
| | 100% | 91% | 80% | 70% | 61% | 54% | 45% | 36% | 30% | 19% | 0% | 0% |
| Formulation C | 2410 | 2206 | 1970 | 423 | 319 | 294 | 253 | 195 | 155 | 109 | 0 | 0 |
| | 100% | 94% | 83% | 70% | 53% | 52% | 47% | 37% | 32% | 23% | 0% | 0% |

TABLE 4
Concentrations of CDCA (mg/L) measured during incubation

| | SI incubation | | | Colonic incubation | | | | | | | | |
|---|---|---|---|---|---|---|---|---|---|---|---|---|
| | 0 h | 1 h | 2 h | 0 h | 1 h | 2 h | 3 h | 4 h | 5 h | 6 h | 19 h | 24 h |
| Control | 2411 | 2250 | 2370 | 538 | 512 | 458 | 392 | 342 | 335 | 331 | 320 | 328 |
| Cholestyramine | 2411 | 388 | 231 | 60 | 51 | 61 | 56 | 45 | 58 | 63 | 66 | 56 |
| | 100% | 17% | 10% | 11% | 10% | 13% | 14% | 13% | 17% | 19% | 21% | 17% |
| Formulation A | 2411 | 2072 | 1874 | 389 | 306 | 274 | 250 | 237 | 207 | 162 | 67 | 79 |
| | 100% | 92% | 79% | 72% | 60% | 60% | 64% | 69% | 62% | 49% | 21% | 24% |
| Formulation B | 2411 | 1936 | 1692 | 354 | 317 | 280 | 230 | 183 | 169 | 175 | 91 | 76 |
| | 100% | 86% | 71% | 66% | 62% | 61% | 59% | 54% | 50% | 53% | 28% | 23% |
| Formulation C | 2411 | 2163 | 2009 | 399 | 305 | 261 | 240 | 216 | 190 | 153 | 77 | 76 |
| | 100% | 96% | 85% | 74% | 60% | 57% | 61% | 63% | 57% | 46% | 24% | 23% |

TABLE 5
Concentrations of DCA (mg/L) measured during incubation

| | SI incubation | | | Colonic incubation | | | | | | | | |
|---|---|---|---|---|---|---|---|---|---|---|---|---|
| | 0 h | 1 h | 2 h | 0 h | 1 h | 2 h | 3 h | 4 h | 5 h | 6 h | 19 h | 24 h |
| Control | 1207 | 1084 | 1147 | 280 | 265 | 242 | 201 | 167 | 141 | 129 | 74 | 68 |
| Cholestyramine | 1207 | 210 | 134 | 18 | 4 | 13 | 14 | 2 | 1 | 1 | 16 | 9 |
| | 100% | 19% | 12% | 6% | 2% | 5% | 7% | 1% | 1% | 1% | 22% | 13% |
| Formulation A | 1207 | 997 | 906 | 206 | 159 | 128 | 103 | 86 | 80 | 43 | 5 | 4 |
| | 100% | 92% | 79% | 74% | 60% | 53% | 51% | 51% | 57% | 33% | 7% | 6% |
| Formulation B | 1207 | 977 | 837 | 188 | 157 | 132 | 105 | 70 | 52 | 58 | 2 | 9 |
| | 100% | 90% | 73% | 67% | 59% | 55% | 52% | 42% | 37% | 45% | 3% | 13% |
| Formulation C | 1207 | 1049 | 981 | 206 | 152 | 121 | 104 | 84 | 86 | 49 | 6 | 2 |
| | 100% | 97% | 86% | 74% | 57% | 50% | 52% | 50% | 61% | 38% | 8% | 3% |

The measured concentrations of the different bile acids in the control sample confirm the effect and extent of microbial salt metabolism in the gut (e.g. deconjugation, dehydrogenation and dehydroxylation), particularly in the colon. A sudden and large decrease of the concentrations of CA, CDCA and DCA in the control sample was observed during the transition of the small intestinal to the colonic incubation.

It can be seen that the three formulations offered a protection of the active compound during the small intestinal incubation. Whereas pure (uncoated) cholestyramine displayed sequestration of CA, 90% sequestration of CDCA and 88% sequestration of DCA already after 2 hours of small intestinal incubation, formulations A, B and C gave rise to much less sequestration of bile salts during this period. After 2 hours in small intestinal incubation, the sequestration of CA, CDCA, and DCA was less than 30%. The sequestration of CA was even less than 20% during this period.

### Example 8

Stability Test

Hard capsules comprising formulation C (250 mg cholestyramine) were stored at 25° C./60% RH during 11 months.

After 0, 3, 6 and 11 months of storage, the capsules were analysed for cholestyramine and water content. Also, the sequestering capacity of the formulation was determined using the assay described in Example 5. After 11 months, the capsules were stored at room temperature and ambient relative humidity. The sequestering capacity of the formulation was then determined once more after approximately 18 months. The results are shown in the table below.

| | | Time (months) | | | | |
|---|---|---|---|---|---|---|
| Analysis | Units | 0 | 3 | 6 | 11 | ~18 |
| Cholestyramine | mg/capsule | 250 | 246 | 245 | | |
| content | % of initial | 100 | 98.4 | 98.0 | | |
| Water content | % | 18.3 | 17.8 | 16.9 | | |
| Sequestration | % | 7 | 10 | 5 | 5 | 4 |
| pH 5.5 (6 h) | | | | | | |
| Sequestration | % | 34 | 35 | 36 | 36 | 39 |
| pH 1 (2 h) + | | | | | | |
| pH 6.8 (4 h) | | | | | | |

1. An oral formulation for targeted delivery of cholestyramine to the colon, comprising:
a) a plurality of extruded and spheronized pellets, each extruded and spheronized pellet comprising at least about 70% w/w cholestyramine and at least about 5% w/w of an acrylate copolymer;
b) a diffusion-controlled inner coating surrounding each extruded and spheronized pellet; and
c) an enteric outer coating.

2. The formulation according to claim 1, wherein the diffusion-controlled inner coating is elastic.

3. The formulation according to claim 1, wherein the diffusion-controlled inner coating comprises poly(ethyl acrylate-co-methyl methacrylate-co-trimethylammonioethyl methacrylate chloride) 1:2:0.2, poly(ethyl acrylate-co-methyl methacrylate-co-trimethylammonioethyl methacrylate chloride) 1:2:0.1, or a combination thereof.

4. The formulation according to claim 1, wherein the enteric outer coating comprises hydroxypropyl methylcellulose acetate succinate.

5. The formulation according to claim 1, wherein the diameter of the uncoated extruded and spheronized pellets is from about 700 to about 1400 μm.

6. (canceled)

7. The formulation according to claim 1, wherein the uncoated extruded and spheronized pellets also comprise microcrystalline cellulose.

8.-11. (canceled)

12. The formulation according to claim 1, wherein the cholestyramine content of the final formulation (on dry weight basis) is at least 50% w/w.

13. (canceled)

14. The formulation according to claim 1, wherein the amount of coating in the final formulation (on dry weight basis) is less than 40% w/w.

15. (canceled)

16. The formulation according to claim 1, wherein the formulation is capable of releasing more than 70% of the cholestyramine in the colon.

17. The formulation according to claims 1, wherein the formulation is capable of releasing less than 30% of the cholestyramine is released in the small intestine.

18. The formulation according to claim 1, wherein the extruded and spheronized pellets exhibit a friability of less than about 2.5% as measured using the European Pharmacopoeia 8.0, test 2.9.7.

19. The formulation according to claim 1, wherein the formulation releases less than about 30% of the cholestyramine after about 6 hours at pH of about 5.5 as measured using the USP Dissolution Apparatus 2 (paddle) Ph. Eur. 2.9.3.

20. The formulation according to claim 1, wherein the formulation exhibits less than about 30% sequestration of cholic acid after about 6 hours at pH of about 5.5 as measured using a USP Dissolution Apparatus 2 (paddle) Ph. Eur. 2.9.3.

21. The formulation according to claim 1, wherein the formulation exhibits greater than about 30% sequestration of cholic acid after about 2 hours at pH of about 1 followed by about 4 hours at pH of about 6.8 as measured using a USP Dissolution Apparatus 2 (paddle) Ph. Eur. 2.9.3.

22. The formulation according to claim 1, wherein the formulation exhibits less than 30% sequestration of cholic acid after about 2 hours at pH of about 1 as measured using a USP Dissolution Apparatus 2 (paddle) Ph. Eur. 2.9.3.

23. The formulation according to claim 1, wherein the formulation exhibits greater than about 30% sequestration of cholic acid after about 2 hours at pH of about 1 followed by about 4 hours at pH of about 7.4 as measured using a USP Dissolution Apparatus 2 (paddle) Ph. Eur. 2.9.3.

24. The formulation according to claim 1, wherein the formulation is contained within a capsule.

25. The formulation according to claim 1, wherein the formulation is contained within a sachet.

26. A method for treating bile acid malabsorption in a patient in need thereof, the method comprising administering to the patient a therapeutically effective amount of an oral formulation comprising:
a) a plurality of extruded and spheronized pellets, each extruded and spheronized pellet comprising cholestyramine and at least about 5% w/w of an acrylate copolymer;
b) a diffusion-controlled inner coating surrounding each extruded and spheronized pellet; and
c) an enteric outer coating.

27. The method according to claim 26, wherein the bile acid malabsorption is the result of ileal disease (Crohn's disease), ileal resection or ileal bypass, the result of overproduction of bile acids or defective feedback inhibition of hepatic bile acid synthesis, or the result of cholecystectomy, vagotomy, small intestinal bacterial overgrowth (SIBO), coeliac disease, pancreatic insufficiency (chronic pancreatitis, cystic fibrosis), pancreatic transplant, radiation enteritis, collagenous colitis, microscopic colitis, lymphocytic colitis, ulcerative colitis or irritable bowel syndrome (IBS-D).

28.-31. (canceled)
